(19) United States
(12) Patent Application Publication
(10) Pub. No.: US 2010/0028356 A1
(43) Pub. Date: Feb. 4, 2010

(54) METHOD OF DIAGNOSING A NEURODEGENERATIVE DISEASE

(75) Inventors: Peter Schofield, New South Wales (AU); John Kwok, New South Wales (AU); Agnes Luty, New South Wales (AU)

Correspondence Address: KNOBBE MARTENS OLSON & BEAR LLP, 2040 MAIN STREET, FOURTEENTH FLOOR, IRVINE, CA, 92614, US

(73) Assignee: POWMRI LTD., Randwick (AU)

(21) Appl. No.: 12/526,481

(22) PCT Filed: Feb. 8, 2008
(86) PCT No.: PCT/AU2008/000164

§ 371 Date: Sep. 18, 2009

Related U.S. Application Data
(60) Provisional application No. 60/900,577, filed on Feb. 8, 2007.

(51) Int. Cl. A61K 39/395; C12Q 1/68; C12N 15/12; C07K 14/47; C07K 16/18; A61P 25/28

(52) U.S. Cl. 424/139.1; 435/6; 536/23.5; 530/350; 530/387.9

(57) ABSTRACT

The present invention provides a method for diagnosing a neurodegenerative disease or for determining the predisposition of a subject to a neurodegenerative disease. In particular, the methods of the present invention comprise detecting a marker linked to map position 9p21, e.g., a marker within an opioid receptor sigma 1 (OPRS1) gene or an expression produce thereof. The present invention also provides a method for identifying new markers that are associated with a neurodegenerative disease. The present invention also provides mutant forms of an OPRS1 gene or an expression product thereof and reagents for detecting those mutations.

### RELATED APPLICATION DATA

This application claims priority from U.S. Ser. No. 60/900,577 filed on Feb. 8, 2007 the entire contents of which are hereby incorporated by reference.

### FIELD OF THE INVENTION

The present invention relates to a method for diagnosing a neurodegenerative disease in a subject and/or for determining the predisposition of a subject to a neurodegenerative disease. In particular, the methods of the present invention comprise detecting a marker that comprises one or more polymorphisms and/or one or more allelic variants and/or one or more mutations linked to map position 9q21, e.g., in an opioid receptor sigma (OPRS) 1 gene or an expression product thereof.

### BACKGROUND OF INVENTION

1. General

The following publications provide conventional techniques of molecular biology. Such procedures are described, for example, in the following texts that are incorporated y reference:
(i) Sambrook, Fritsch & Maniatis, Molecular Cloning: A Laboratory Manual, Cold Spring Harbor Laboratories, New York, Second Edition (1989), whole of Vols I, II, and III;
(ii) DNA Cloning: A Practical Approach, Vols. I and II (D. N. Glover, ed., 1985), IRL Press, Oxford, whole of text;
(iii) Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
(iv) Nucleic Acid Hybridization: A Practical Approach (B. D. Hames & S. J. Higgins, eds., 1985) IRL Press, Oxford, whole of text;
(v) Perbal, B., A Practical Guide to Molecular Cloning (1984);
(vi) Methods In Enzymology (S. Colowick and N. Kaplan, eds., Academic Press, Inc.), whole of series;
(vii) J. F. Ramalho Ortigão, “The Chemistry of Peptide Synthesis” In: Knowledge database of Access to Virtual Laboratory website (Interactiva, Germany);
(viii) Sakakibara, D., Teichman, J., Lien, E. Land Fenichel, R. L. (1976). Biochem. Biophys. Res. Commun. 73 336-342
(ix) Merrifield, R. B. (1963). J. Am. Chem. Soc. 85, 2149-2154.
(x) Handbook of Experimental Immunology, Vols. I-IV (D. M. Weir and C. C. Blackwell, eds., 1986, Blackwell Scientific Publications).

2. Description of the Related Art

Neurodegenerative diseases are a group of disorders characterized by changes in normal neuronal function, leading in the majority of cases to neuronal dysfunction and even cell death. Currently, it is estimated that there are in excess of one hundred neurodegenerative diseases. However, we still have little understanding of the etiological cause of these diseases. The most consistent risk factor for the development of a neurodegenerative disease, such as, for example, dementia, e.g., Alzheimer's disease or frontotemporal lobar dementia is age (Tanner, Neurol. Clin. 10: 317-329, 1992). For example, such diseases are more prevalent in aged or aging persons, with a doubling of risk every five years after the age of 65.

One of the most common forms of neurodegenerative disease is dementia. Dementia is a class of neurodegenerative diseases characterized by more rapid progressive decline of cognitive function in a subject than is expected to occur as a result of normal aging. Generally, dementia is caused by neurological damage, disease and/or degeneration. For example, dementia is known to be caused by diseases such as, for example, Alzheimer's disease, frontotemporal dementia, dementia with lewy bodies, frontotemporal lobar degeneration and prion diseases. As discussed further, below, dementia is generally observed in elderly subjects (i.e., 65 years of age or older). In this respect, in USA approximately 4 million to 5 million people suffer from a form of dementia.

Over the past century, the growth rate of the population aged 65 and beyond in industrialized countries has far exceeded that of the population as a whole. Accordingly, it is anticipated that, over the next generations, the proportion of elderly citizens will double, and, with this, the proportion of persons suffering from dementia.

### Dementia

Notwithstanding that dementia usually occurs in subjects over the age of 65, early onset or presenile dementia is observed in subjects under the age of 65. In this respect, a Health Retirement Study conducted in USA by Institute for Social Research at the University of Michigan found that approximately 480,000 subjects in USA suffered from some form of presenile dementia.

Presenile dementia is generally caused by diseases, such as, for example, Alzheimer's disease, Parkinson's disease, frontotemporal dementia, dementia with lewy bodies and prion diseases. However, in presenile dementia, the onset of detectable cognitive symptoms occurs before the age of 65.

### Causes of Dementia

The most common and most studied forms of dementia are Alzheimer's disease and frontotemporal dementia/frontotemporal lobar degeneration (Neary et al., Neurology 51: 1546-1554, 1998). Currently, it is estimated that there are 4.5 million cases of Alzheimer's disease in the US alone and that between about 12% and about 16% of patients with degenerative dementia. It is estimated that in the period from 2001 to 2010 an additional 1.5 million Alzheimer's disease cases will be diagnosed in the US, while currently there are approximately 480 new cases of Parkinson's disease per million people per year diagnosed. Alzheimer's disease alone is the third most expensive disease in the United States, costing approximately US$100 billion each year for therapy and/or care of sufferers.

### Alzheimer's Disease

Alzheimer's disease is a complex multigenic neurological disorder characterized by progressive impairments in memory, behavior, language, and visuo-spatial skills, ending ultimately in death. Hallmark pathologies of Alzheimer's disease include granulovascular neuronal degeneration, extracellular neuritic plaques with β-amyloid deposits, intracellular neurofibrillary tangles and neurofibrillary degeneration, synaptic loss, and extensive neuronal cell death. It is now known that these histopathologic lesions of Alzheimer's disease correlate with the dementia observed in many elderly people.

Alzheimer's disease is commonly diagnosed using clinical evaluation including, physical and psychological assessment, an electroencephalography (EEG) scan, a computerized tomography (CT) scan and/or an electrocardiogram. These forms of testing are performed to eliminate some possible causes of dementia other than Alzheimer's disease, such as, for example, a stroke. Following elimination of other possible causes of dementia, Alzheimer's disease is diagnosed. Accordingly, current diagnostic approaches for Alzheimer's disease are not only unreliable and subjective, they do not predict the onset of the disease. Rather, these methods merely diagnose the onset of dementia of unknown cause, following onset.

Furthermore, not all causes of dementia are easily detectable by methods currently used for the diagnosis of Alzheimer's disease. Accordingly, a subject that has suffered an ischemic, metabolic, toxic, infectious or traumatic insult to the brain may also present with dementia, and, as a consequence, be incorrectly diagnosed with Alzheimer's disease. In fact, the NIH estimates that up to 45% of subjects diagnosed with Alzheimer's disease actually suffer from another form of dementia.

Genetic studies of subjects with a family history of Alzheimer's disease indicate that mutations in genes, such as, for example, amyloid precursor protein, presenillin-1 or presenillin-2 cause early onset forms of this disease. However, these forms of Alzheimer's disease represent less than 5% of total cases of the disease.

Studies to identify polymorphisms and alleles that confer susceptibility to Alzheimer's disease have identified a large number of polymorphisms and mutations (reviewed in Rocchi et al., Brain Res. Bull., 61: 1-24, 2003). The most widely studied of these is the ε4 isoform of the apolipoprotein E gene. A number studies have shown an association between apolipoprotein E ε4 (ApoE-ε4) and late onset familial and sporadic forms of Alzheimer's disease (for example, Corder et al., Science 261: 261-263, 1993). However, less than 50% of non-familial Alzheimer's disease sufferers are carriers of the ApoE-ε4 isoform (Corder et al., Science 261: 261-263, 1993).

### Frontotemporal Lobar Degeneration

Frontotemporal lobar degeneration (FTLD) is the third most common neurodegenerative disease resulting in dementia after Alzheimer's disease and dementia with Lewy bodies. Pathologically, FTLD is characterized by degeneration of neurons in the superficial frontal cortex and anterior temporal lobes. FTLD is a pathologically heterogeneous disorder categorized into cases without detectable intra or inter-cellular inclusions known as dementia lacking distinctive histopathology, cases with tau-positive pathology also known as tauopathies, and the most frequently recognized TDP-43 proteinopathies (Cairns et al., Acta Neuropathol., 114: 5-22, 2007). TDP-43 is a major protein component of ubiquitin-immunoreactive, tau- and α-synuclein-negative inclusions found in most sporadic and familial cases of FTLD. Approximately 26% of FTLD patients have intracellular deposits of diffuse beta-amyloid positive plaques (Mann et al., Neurosci. Letters 304: 161-164, 2001).

Patients suffering from FTLD generally develop several clinical presentations characterized by changes and personality and behavior, including a decline in manners and social skills representative of frontotemporal degeneration, and language disorders of expression (progressive aphasia) and comprehension (semantic dementia) (Neary et al., supra). However, in some cases amnesia is the presenting feature of FTLD (Graham et al., Brain, 128: 597-605, 2005).

Given the pronounced variation in initial presentation of FTLD and the early prominence of behavioral symptoms common to other neurological disorders, misdiagnosis is a common problem for patients and their families (Greicius et al., Journal of Neurology Neurosurgery and Psychiatry, 72: 691-700, 2002).

Approximately 40% of cases of FTLD are familial, indicating a significant genetic contribution to this disease (Rosso et al., Brain, 126: 2016-2022, 2003). Causal mutations were first identified in FTLD with Parkinsonism in the gene encoding microtubule associated protein tau (MAPT) on Chromosome 17 (Hutton et al., Nature, 393: 702-705, 1998). More recently, mutations in the progranulin (PGN) gene on chromosome 17 have also been identified (Baker et al., Nature, 442: 916-919, 2006). Mutations in the charged vesicular body protein 2 (CHMP2B) gene on Chromosome 3 have been associated with the rare form of TDP-43 negative FTLD-U (Skibinski et al., Nature Genetics, 37: 806-808, 2005). Mutations in the Valosin Containing Protein (VCP) gene have also been reported in a rare form of FTLD which includes inclusion body myopathy and Paget disease of the bone (Watt et al., Nature Genetics, 36: 377-381, 2004). However, these mutations do not account for the majority of familial cases of frontotemporal dementia, and are rarely observed in sporadic frontotemporal dementia (Houlden et al. Ann Neurol, 46:243-8, 1999).

### Motor Neuron Disease

Motor neuron disease is generally characterized by degeneration of the upper and/or motor neurons. Motor neuron diseases are a class of diseases including amyotrophic lateral sclerosis, spinal muscular atrophy and spinal and bulbar muscular atrophy (SBMA, or Kennedy's disease). The most common form of motor neuron disease is ALS, which is characterized by degeneration of the upper and lower motor neurons, leading to progressive muscle atrophy and wasting, weakness and spasticity. Ultimately, ALS patients suffer from profound global paralysis and often die prematurely as a result of respiratory failure.

Approximately 10% of motor neuron cases have a positive family history (Strong et al., Can. J. Neurol. sci., 18: 45-58, 1991). Mutations and polymorphisms associated with or causative of motor neuron disease have been identified in several genes, e.g., superoxide dismutase (SOD1) gene on chromosome 21q22 (Rosen et al., Nature, 362: 59-62, 1993), dynactin (DCTN1) on Chromosome 2p13 (Nishimura et al., Am. J. Hum. Genet., 75: 822-831, 2004), and vesicle trafficking protein (VAPB) on chromosome 20q13 (Puls et al., Nat. Genet., 33: 455-456, 2003). Linkage to chromosomes 15q15-q22, 18q and 16q, have also been reported. However, all of the mutations identified to date account for less than half of inherited cases of motor neuron disease. Accordingly, a significant proportion of subjects that will develop familial motor neuron disease still go undiagnosed prior to development of clinical symptoms.

Based on the discussion herein it is clear that there is a need to develop improved diagnostic methods for determining a predisposition to development of a neurodegenerative disease in a subject, and for the early diagnosis of neurodegenerative disease, e.g., presenile dementia. There is also a need in the art for molecular markers of neurodegenerative disease, to thereby facilitate the production of a rapid, reliable and non-invasive diagnostic/prognostic assays for determining a predisposition to development of neurodegenerative disease in a subject, and for the early diagnosis of neurodegenerative disease.

### SUMMARY OF INVENTION

In work leading up to the present invention the inventors sought to identify mutations and/or polymorphisms that are significantly associated with development of a neurodegenerative disease for use in a new diagnostic and/or prognostic method.

As exemplified herein, the inventors studied mutations and/or polymorphisms associated with two common dementias, viz., early onset Alzheimer's disease and frontotemporal lobar dementia (FTLD) to identify genetic and/or biochemical markers for use in diagnostic and/or predictive assays. As will be apparent to the skilled artisan from the foregoing description, early onset Alzheimer's disease and FTLD are distinct forms of dementia. Accordingly, any marker described herein that is identified in subjects suffering from either Alzheimer's disease or frontotemporal dementia, or both is suitable for the diagnosis or prediction of dementia generally. The present inventors also identified mutations in subjects suffering from dementia and motor neuron disease and in subjects suffering from motor neuron disease. These diseases represent a diverse range of neurodegenerative disease. Accordingly, the markers described herein that are identified in subjects suffering from dementia and/or motor neuron disease are suitable for the diagnosis or prediction of neurodegenerative disease generally. As exemplified herein, the present inventors have identified a neurodegenerative disease susceptibility locus linked to map position 9p21-9q21, by conducting linkage analysis of these dementias, e.g., a locus linked to map position 9p21.1-9p21.2.

Further analysis by the inventors identified at least one nucleic acid change in the opioid receptor sigma 1 (OPRS1) gene in subjects suffering from neurodegenerative disease, e.g., early onset Alzheimer's disease or FTLD and/or motor neuron disease. For example, the inventors identified a non-polymorphic nucleotide change in the 3′-untranslated region of the OPRS1 gene in subjects suffering from dementia that was not observed in control subjects. The inventors also found that this nucleotide change is associated with enhanced expression of the OPRS1 gene in subjects suffering from neurodegenerative disease.

The present inventors then screened a panel of 266 presenile dementia patients and panels of subjects suffering from motor neuron disease, and detected additional nucleotide changes located within the OPRS1 gene. For example, the inventors identified at least 5 mutations associated with or causative of motor neuron disease and/or at least 5 mutations associated with or causative of early onset dementia and/or at least 4 mutations associated with or causative of FTLD and/or at least one mutation associated with or causative of early onset Alzheimer's disease. For example, the inventors identified two nucleotide changes in introns of the OPRS1 gene that alters splicing of mRNA encoded therefrom, and reduces levels of normally spliced OPRS1 mRNA. One of these nucleotide changes is located within intron 2 of the OPRS1 gene, and occurs within the binding site of two splicing factors, hnSNPF/H and SC35 in the OPRS1 transcript. The inventors also identified a nucleotide substitution and a nucleotide insertion in the OPRS1 promoter region that is associated with increased expression of OPRS1.

The inventors also identified a mutation in patients suffering from a neurodegenerative disease, e.g., early onset Alzheimer's disease subjects that results in an alanine to valine substitution at amino acid position 4 of the OPRS1 protein. This mutation is associated with increased levels of gamma-secretase, a protein that cleaves β-amyloid to form the Aβ peptide identified in plaques in subjects suffering from Alzheimer's disease. Such a mutation additionally provides the basis of a method for diagnosing a neurodegenerative disease, e.g., Alzheimer's disease, e.g., early onset Alzheimer's disease or for determining the predisposition of a subject to Alzheimer's disease, e.g., early onset Alzheimer's disease.

Each of the nucleotide changes identified by the present inventors in the OPRS1 gene and the amino acid changes in the OPRS1 protein provide the basis for a method for diagnosing a neurodegenerative disease in a subject or determining a predisposition of a subject to developing a neurodegenerative disease or for determining the risk of a subject to developing a neurodegenerative disease.

### Specific Embodiments

The scope of the invention will be apparent from the claims as filed with the application that follow the examples. The claims as filed with the application are hereby incorporated into the description. The scope of the invention will also be apparent from the following description of specific embodiments and/or detailed description of preferred embodiments.

The present invention provides a method for diagnosing a neurodegenerative disease in a subject or determining the predisposition of a subject to developing a neurodegenerative disease or determining an increased risk of a subject developing dementia neurodegenerative disease, the method comprising detecting in a sample from the subject a marker linked to chromosome 9p21-9q21 of the human genome, wherein detection of said marker is indicative of a neurodegenerative disease or a predisposition to a neurodegenerative disease or an increased risk of a subject developing neurodegenerative disease.

Preferably, the marker is linked to map position 9p21.1-9p21.2.

In one example, the marker linked to map position 9p21-9q21 is located between or comprises the microsatellite markers designated D9S161 (SEQ ID NO: 1) and D9S175 (SEQ ID NO: 2). For example, the marker linked to map position 9p21-9q21 of the human genome is located between or comprises the microsatellite markers designated D9S161 (SEQ ID NO: 1) and D9S273 (SEQ ID NO: 3). For example, the marker linked to map position 9p21-9q21 of the human genome is linked to and/or comprises the microsatellite marker designated D9S1817 (SEQ ID NO: 4) and/or D9S163 (SEQ ID NO: 14) and/or D9S1845 (SEQ ID NO: 15) and/or D9S1118 (SEQ ID NO: 16) and/or D9S319 (SEQ ID NO: 17). Preferably, the marker linked to map position 9p21-9q21 of the human genome is linked to and/or comprises the microsatellite marker designated D9S319 (SEQ ID NO: 17)

As used herein, the terms “linked” and “map to” shall be taken to refer to a sufficient proximity between a marker and nucleic acid comprising all or part of map position 9p21-9q21 of the human genome or an expression product thereof to permit said linked nucleic acid to be useful for diagnosing a neurodegenerative disease in a subject or a predisposition to dementia or an increased risk of developing a neurodegenerative disease. Those skilled in the art will be aware that for linked nucleic acid to be used in this manner, it must be sufficiently close to map position 9p21 so as to be in linkage or for there to be a low recombination frequency between the linked nucleic acid and map position 9p21-9q21. Preferably, the linked nucleic acid and the locus are less than about 25 cM apart, more preferably less than about 10 cM apart, even more preferably less than about 5 cM apart, still more preferably less than about 3 cM apart and still more preferably less than about 1 cM apart.

The present invention also provides a method for diagnosing a neurodegenerative disease in a subject or determining the predisposition of a subject to developing a neurodegenerative disease or determining an increased risk of a subject developing a neurodegenerative disease, the method comprising detecting in a sample from the subject a marker within an opioid receptor sigma 1 (OPRS1) gene or an expression product thereof that is associated with or linked or causative of a neurodegenerative disease, wherein detection of said marker is indicative of a neurodegenerative disease or a predisposition to a neurodegenerative disease or an increased risk of developing a neurodegenerative disease.

For the purposes of nomenclature, a human OPRS1 gene comprises a nucleotide sequence set forth in SEQ ID NO: 13 and/or capable of encoding a sequence set forth in SEQ ID NO: 5. Preferably, a human OPRS1 gene comprises a sequence at least about 80% identical to the sequence set forth in SEQ ID NO: 13 and/or a sequence encoding a nucleic acid comprising a sequence at least about 80% identical to the sequence set forth in SEQ ID NO: 5. More preferably, the nucleic acid comprises a sequence at least about 85% identical to the sequence set forth in SEQ ID NO: 13 or at least about 90% to the sequence set forth in SEQ ID NO: 13 or at least about 95% identical to the to the sequence set forth in SEQ ID NO: 13. Alternatively, or in addition, the nucleic acid comprises a sequence that encodes a sequence at least about 85% identical or at least about 90% identical or at least about 95% identical to the sequence set forth in SEQ ID NO: 5.

In one example, a marker associated with or causative of a neurodegenerative disease occurs within an OPRS1 genomic gene. A genomic gene of OPRS1 shall be understood to include the coding region of a OPRS1 protein (e.g., codons required to encode any isozyme of OPRS1) in addition to intervening intronic sequences in addition to regulatory regions that control the expression of said gene, e.g., a promoter or fragment thereof and/or a 5′ untranslated region and/or a 3′ untranslated region.

As used herein, the term “neurodegenerative disease” shall be taken to mean a disease that is characterized by neuronal cell death. The neuronal cell death observed in a neurodegenerative disease is often preceded by neuronal dysfunction, sometimes by several years. Accordingly, the term “neurodegenerative disease” includes a disease or disorder that is characterized by neuronal dysfunction and eventually neuronal cell death. Often neurodegenerative diseases are also characterized by increased gliosis (e.g., astrocytosis or microgliosis) in the region/s of neuronal death. Cellular events observed in a neurodegenerative disease often manifest as a behavioral change (e.g., deterioration of thinking and/or memory) and/or a movement change (e.g., tremor, ataxia, postural change and/or rigidity). Examples of neurodegenerative disease include, for example, FTLD, Alzheimer's disease, amyotrophic lateral sclerosis, ataxia (e.g., spinocerebellar ataxia or Friedreich's Ataxia), Creutzfeldt-Jakob Disease, a polyglutamine disease (e.g., Huntington's disease or spinal bulbar muscular atrophy), Hallervorden-Spatz disease, idiopathic torsion disease, Lewy body disease, multiple system atrophy, neuroanthocytosis syndrome, olivopontocerebellar atrophy, Parkinson's disease, Pelizaeus-Merzbacher disease, Pick's disease, progressive supranuclear palsy, syringomyelia, torticollis, spinal muscular atophy or a trinucleotide repeat disease (e.g., Fragile X Syndrome). Preferably, the neurodegenerative disease is a neurodegenerative disease associated with aberrant OPRS1 expression and/or activity.

Preferably, the neurodegenerative disease is a dementia. As used herein, the term “dementia” shall be taken to mean a neurodegenerative disease is characterized by chronic loss of mental capacity, particularly progressive deterioration of thinking and/or memory and/or behavior and/or personality and/or motor function, and may also be associated with psychological symptoms such as depression and apathy. In this respect, dementia is not caused by, for example, a stroke, an infection or a head trauma. Examples of dementia include, for example, an Alzheimer's disease, vascular dementia, dementia with Lewy bodies and frontotemporal lobar dementia, amongst others.

In one example, the method of the present invention diagnoses presenile dementia and/or determines the predisposition of a subject to presenile dementia and/or determines the risk of a subject to presenile dementia. In this respect, the term “presenile dementia” is understood in the art to mean dementia characterized by the onset of clinically detectable symptoms before a subject is 65 years of age.

In one example, the dementia is an Alzheimer's disease or FTLD. By “an Alzheimer's disease” is meant a neurological disorder characterized by progressive impairments in memory, behavior, language and/or visuo-spatial skills. Pathologically, an Alzheimer's disease is characterized by neuronal loss, gliosis, neurofibrillary tangles, senile plaques, Hirano bodies, granulovacuolar degeneration of neurons, amyloid angiopathy and/or acetylcholine deficiency. The term “an Alzheimer's disease” shall be taken to include early onset Alzheimer's disease (e.g., with an onset of detectable symptoms occurring before a subject is 65 years of age) or a late onset Alzheimer's disease (e.g., with an onset later then, or in, the sixth decade of life). Preferably, the Alzheimer's disease is an early onset Alzheimer's disease.

In one example, the Alzheimer's disease is an early onset Alzheimer's disease. For example, the present inventors have identified at least one nucleotide change (a thymidine at a position corresponding to nucleotide position 1005 of SEQ ID NO: 7) in the OPRS1 gene that occurs in subjects suffering from early onset Alzheimer's disease.

For example, the Alzheimer's disease is a plaque predominant Alzheimer's disease. As used herein, the term “plaque predominant Alzheimer's disease” shall be taken to mean a variant form of Alzheimer's disease characterized by numerous senile plaques in the relative absence of neurofibrillary tangles.

In another example, the disease is a motor neuron disease. As used herein, the term “motor neuron disease” shall be taken to mean a disease characterized by dysfunction and/or death of motor neurons, e.g., upper motor neurons and/or lower motor neurons.

Generally, a motor neuron disease presents as muscle weakness and atrophy, with the weakness often presenting in the limbs and/or as difficulty swallowing. As motor neuron disease progresses an affected subject often develops difficulty walking and lifting objects, and eventually difficulty breathing. Exemplary motor neuron diseases include amyotrophic lateral sclerosis (ALS) and spinal muscular atrophy (SMA). Preferably, the motor neuron disease is ALS.

As used herein, the term “marker” shall be taken to mean a nucleic acid that comprises a nucleotide sequence associated with or causative of a neurodegenerative disease and/or a nucleotide sequence that occurs in a subject suffering from dementia but does not occur in a subject that does not suffer from dementia.

Alternatively, or in addition, the marker is linked to a polymorphism or nucleotide change in a genome wherein said polymorphism or nucleotide change is associated with dementia. For example, a marker occurs within any region of an OPRS1 genomic gene, including an exon or an intron or a promoter region or an enhancer region or a 3′ untranslated region.

In those methods described herein according to any embodiment comprising detecting a marker in a region of a genome that is transcribed or that controls transcription, the term “marker” shall also be taken to mean an expression product of a gene or an allele of OPRS1 that is associated with dementia. For example, the marker comprises or is within a pre-mRNA molecule, a 5′capped mRNA, a polyadenylated mRNA and/or a mature or processed mRNA.

In those methods described herein according to any embodiment comprising detecting a marker in a region of a genome encoding a polypeptide, those skilled in the art will appreciate that the term “marker” also means a peptide, polypeptide or protein that comprises an amino acid sequence encoded by an allele of an OPRS1 gene that is associated with or linked to or causative of a neurodegenerative disease.

As used herein, the term “associated with a neurodegenerative disease” shall be taken to mean that the detection of a marker is significantly correlated with the development of neurodegenerative disease in a subject or that the absence of a marker is significantly correlated with the development of a neurodegenerative disease. For example, a marker occurs in a subject or is detectable in a subject that suffers from neurodegenerative disease and does not occur in a subject or is not detectable in a subject that does not suffer from neurodegenerative disease. Alternatively, or in addition, detection of a marker associated with neurodegenerative disease is significantly correlated with the development of neurodegenerative disease in a subject or that the absence of a marker is significantly correlated with the development of neurodegenerative disease. For example, in the case of a marker that is positively associated with a disease is a polymorphism the detection of that marker is associated with the development of neurodegenerative disease. As used herein, the term “polymorphism” shall be taken to mean a difference in the nucleotide sequence of a specific site or region of the genome of a subject that occurs in a population of individuals, wherein one form of the polymorphism is associated with a neurodegenerative disease. Exemplary polymorphisms include a simple sequence repeat or microsatellite marker, e.g. in which the length of the marker varies between individuals in a population or a simple nucleotide polymorphism. The skilled artisan will understand that a simple nucleotide polymorphism is a small change (e.g., an insertion, a deletion, a transition or a transversion) that occurs in a genome of a population of subjects. For example, a simple nucleotide polymorphism comprises or consists of an insertion or deletion or transversion of one, or two or three or five, or ten or twenty nucleotides in the genome of a subject. Preferably, the polymorphism is a single nucleotide polymorphism (SNP). In one example, a polymorphism is significantly correlated with the development of neurodegenerative disease in a plurality of subjects. E.g., the polymorphism is significantly correlated with the development of dementia in a plurality of unrelated subjects.

Whilst the present invention contemplates any marker in an OPRS-1 nucleic acid or polypeptide, it is preferred that the marker comprises or consists of a mutation within an OPRS-1 gene or expression product. By “mutation” is meant a permanent, transmissible change in nucleotide sequence of the genome of a subject and optionally, an expression product thereof that alters the level of expression or activity of native OPRS1 polypeptide thereby causing a neurodegenerative disease. Examples of mutations include an insertion of one or more new nucleotides or deletion of one or more nucleotides or substitute of one or more existing nucleotides with different nucleotides. Such a mutation may also lead to a change in the amino acid of an OPRS1 polypeptide, e.g., altering the activity of an OPRS1 polypeptide. A “mutation” is a difference in the sequence of an OPRS1 gene or an expression product thereof in a subject that suffers from a neurodegenerative disease and that does not occur in a subject that does not suffer from a neurodegenerative disease, for example, in a population of individuals that do not suffer from a neurodegenerative disease.

As used herein, the term “predisposition to neurodegenerative disease” shall be taken to mean that a subject comprising a marker detected by a method as described herein according to any embodiment is susceptible to developing a neurodegenerative disease or is more likely to develop neurodegenerative disease than a normal individual or a normal population of individuals. In this regard, a marker that is indicative of a predisposition to a neurodegenerative disease may itself cause the disease or disorder or, alternatively, be correlated with the development of a neurodegenerative disease.

For example, a marker comprises a thymidine at a position corresponding to nucleotide position 1005 of SEQ ID NO: 7. Alternatively, or in addition a marker comprises an adenosine at a position corresponding to nucleotide position 80 of SEQ ID NO: 5. Alternatively, or in addition, a marker comprises a thymidine at a position corresponding to nucleotide position 85 of SEQ ID NO: 5. Alternatively, or in addition a marker comprises an adenosine at a position corresponding to nucleotide position 626 of SEQ ID NO: 5. Alternatively, or in addition, a marker comprises a guanine at a position corresponding to nucleotide position 699 of SEQ ID NO: 8 and at a position corresponding to nucleotide position 700 of SEQ ID NO: 9. Alternatively, or in addition, a marker comprises a guanine at a position corresponding to position 2080 of SEQ ID NO: 13. Alternatively, or in addition, a marker comprises an adenosine at a position corresponding to position 2080 of SEQ ID NO: 13. Alternatively, or in addition, a marker comprises a cytosine at a position corresponding to position 2092 of SEQ ID NO: 13. Alternatively, or in addition, a marker comprises a thymidine at a position corresponding to position 2092 of SEQ ID NO: 13. Alternatively, or in addition, a marker comprises a guanine at a position corresponding to position 2583 of SEQ ID NO: 13. Alternatively, or in addition, a marker comprises a thymidine at a position corresponding to position 2583 of SEQ ID NO: 13. Alternatively, or in addition, a marker comprises a cytosine at a position corresponding to position 4020 of SEQ ID NO: 13. Alternatively, or in addition, a marker comprises a thymidine at a position corresponding to position 4020 of SEQ ID NO: 13. Alternatively, or in addition, a marker comprises a guanine at a position corresponding to position 4191 of SEQ ID NO: 13. Alternatively, or in addition, a marker comprises a thymidine at a position corresponding to position 4191 of SEQ ID NO: 13.

In one example, a marker comprises an adenosine at a position corresponding to position 2080 of SEQ ID NO: 13. Alternatively, or in addition, a marker comprises a thymidine at a position corresponding to position 2092 of SEQ ID NO: 13. Alternatively, or in addition, a marker comprises a thymidine at a position corresponding to position 2583 of SEQ ID NO: 13. Alternatively, or in addition, a marker comprises a thymidine at a position corresponding to position 4020 of SEQ ID NO: 13. Alternatively, or in addition, a marker comprises a thymidine at a position corresponding to position 4191 of SEQ ID NO: 13.

Alternatively, or in addition, a marker is associated with or causes alternative splicing of an OPRS1 mRNA. As used herein, the term “alternative splicing” shall be taken to mean the insertion or removal of exons into/from an OPRS1 mRNA. Accordingly, an alternatively spliced OPRS1 mRNA comprises additional exons, or lack exons (e.g., nucleotides) compared to the sequence of an OPRS1 cDNA set forth in SEQ ID NO: 2. In one embodiment, the presence of a marker that is associated with alternative splicing of an OPRS1 mRNA is correlated with modulated levels of alternatively spliced OPRS1 mRNA. For example, the marker occurs within a binding site of a splicing factor, such as, for example, hnSNPF/H and/or SC35, thereby modulating the level of splicing of an OPRS1 transcript. Accordingly, the level of a specific splice form of OPRS1 is increased or decreased when the marker is present and is useful for detecting a marker associated with a disease or disorder. Exemplary markers associated with or causative of alternative splicing of an OPRS1 transcript comprises a thymine at a position corresponding to nucleotide position 2583 of SEQ ID NO: 13 or nucleotide position 2576 of SEQ ID NO: 13 or an adenosine at a position corresponding to nucleotide position 2254 of SEQ ID NO: 13, or an adenosine at a position corresponding to nucleotide position 2255 of SEQ ID NO: 13, or an adenosine at a position corresponding to nucleotide position 2257 of SEQ ID NO: 13, or an adenosine at a position corresponding to nucleotide position 2792 of SEQ ID NO: 13. These markers are also associated with a reduced level of a native OPRS1 expression product, e.g., a reduced level of a transcript comprising a sequence set forth in SEQ ID NO: 5.

In another example, a marker is associated with increased expression of an OPRS1 transcript. For example, the marker comprises a thymine at a position corresponding to nucleotide position 4191 of SEQ ID NO: 13 or an adenosine at a position corresponding to nucleotide position 4187 of SEQ ID NO: 3.

Alternatively, or in addition, a marker comprises a valine at a position corresponding to amino acid position 4 of SEQ ID NO: 3. Alternatively, or in addition, a marker comprises a valine at a position corresponding to amino acid position 184 of SEQ ID NO: 3.

In one example, a method described herein is for diagnosing a presenile dementia or determining a predisposition to a presenile dementia or determining an increased risk of developing a presenile dementia. Preferably, such a method detects any one or more markers selected from the group consisting of an adenosine at a position corresponding to position 2080 of SEQ ID NO: 13 or position 80 of SEQ ID NO: 5, a valine at apposition corresponding to amino acid residue 4 of SEQ ID NO: 6, a thymidine at a position corresponding to position 2092 of SEQ ID NO: 13 or position 85 of SEQ ID NO: 5, a thymidine at a position corresponding to position 25783 of SEQ ID NO: 13, a thymidine at a position corresponding to nucleotide position 4020 of SEQ ID NO: 13 or position 626 of SEQ ID NO: 5, a thymidine at a position corresponding to position 4191 of SEQ ID NO: 13 or position 1005 of SEQ ID NO: 7, a guanine at a position corresponding to nucleotide position 30 of SEQ ID NO: 5 or nucleotide position 2030 of SEQ ID NO: 13, a cytosine at a position corresponding to nucleotide position 545 of SEQ ID NO: 5 or nucleotide position 3939 of SEQ ID NO: 13, and an adenosine at a position corresponding to nucleotide position 4187 of SEQ ID NO: 13 and an adenosine at a position corresponding to nucleotide position 729 of SEQ ID NO: 13.

In another example, a method described herein is for diagnosing a presenile dementia or determining a predisposition to a presenile dementia or determining an increased risk of developing a late onset dementia. Preferably, such a method detects an adenosine at a position corresponding to nucleotide position 2576 of SEQ ID NO: 13.

In one example, of the invention a marker is associated with a motor neuron disease. for example, a marker comprises an adenosine at a position corresponding to nucleotide position 2070 of SEQ ID NO: 13 or an adenosine at a position corresponding to nucleotide position 2254 of SEQ ID NO: 13, or an adenosine at a position corresponding to nucleotide position 2255 of SEQ ID NO: 13, or an adenosine at a position corresponding to nucleotide position 2257 of SEQ ID NO: 13, or an adenosine at a position corresponding to nucleotide position 2792 of SEQ ID NO: 13, a thymidine at nucleotide position 141 of SEQ ID NO: 5. In another example, the marker comprises a serine at a position corresponding to amino acid residue 23 of SEQ ID NO: 6.

In the case of a nucleic acid marker associated with a neurodegenerative disease, the marker is preferably detected by hybridizing a nucleic acid probe comprising the sequence of the marker to a marker linked to nucleic acid in a sample from a subject under moderate to high stringency hybridization conditions and detecting the hybridization using a detection means, wherein hybridization of the probe to the sample nucleic acid indicates that the subject suffers from a neurodegenerative disease or a has a predisposition to a neurodegenerative disease or has an increased risk of developing a neurodegenerative disease. For example, the detection means is a nucleic acid hybridization or amplification reaction, such as, for example, a polymerase chain reaction (PCR).

Not only is such a method useful for, for example, detecting a specific polymorphism or mutation in a sample from a subject, but also for detecting a marker in an expression product of an OPRS1 gene, for example, an alternate splice form of an OPRS1 transcript. In this respect, the method of the invention as described herein according to any embodiment comprises detecting a modified level of an alternate splice form encoded by an OPRS1 gene.

At least two of the mutations identified by the present inventors are also associated with modified expression of OPRS1. Accordingly, a subject at risk of developing dementia or that suffers from dementia may equally be determined by detecting a modified level of an OPRS1 expression product in a sample from the subject. In one example, such a method comprises detecting a reduced level of an OPRS1 expression product. In another example, such a method comprises detecting an enhanced level of an OPRS1 expression product. Suitable methods for determining the level of an OPRS1 expression product will be apparent to the skilled person and includes PCR or a variant thereof or an immunoassay, such as is listed above. For example, an enhanced or reduced level of an OPRS1 transcript is detected by performing a process comprising:
(i) determining the level of the OPRS1 transcript in a sample from the subject;
(ii) determining the level of the OPRS1 transcript in a suitable control sample,
wherein an enhanced or reduced level of the OPRS1 transcript at (i) compared to (ii) is indicative of a neurodegenerative disease or a predisposition to a neurodegenerative disease or an increased risk of developing a neurodegenerative disease.

Alternatively, the marker is within an OPRS1 polypeptide. Such a marker is detected, for example, by contacting a biological sample derived from a subject with an antibody or ligand capable of specifically binding to said marker for a time and under conditions sufficient for an antibody/ligand complex to form or a ligand/ligand complex to form and then detecting the complex wherein detection of the complex indicates that the subject being tested suffers from a neurodegenerative disease or a has a predisposition to a neurodegenerative disease or has an increased risk of developing a neurodegenerative disease. A suitable method for detecting the complex will be apparent to the skilled person and includes, for example, an enzyme-linked immunosorbent assay (ELISA), a fluorescence-linked immunosorbent assay (FLISA) an enzyme immunoassay (EIA) or a radioimmunoassay (RIA).

For example, the OPRS1 polypeptide is encoded by an alternatively spliced OPRS1 transcript and/or comprises a valine at a position corresponding to amino acid residue 4 of SEQ ID NO: 6.

In one example of the invention, detecting an enhanced or reduced level of the OPRS1 polypeptide comprises performing a process comprising:
(i) determining the level of the OPRS1 polypeptide in a sample from the subject;
(ii) determining the level of the OPRS1 polypeptide in a suitable control sample,
wherein an enhanced or reduced level of the OPRS1 polypeptide at (i) compared to (ii) is indicative of a neurodegenerative disease or a predisposition to a neurodegenerative disease or an increased risk of developing a neurodegenerative disease.

A suitable control sample will be apparent to the skilled artisan and includes:
(i) a sample from a normal subject;
(ii) a sample from a healthy subject;
(iii) a data set comprising measurements of the level of hybridization or complex in samples from a plurality of normal subjects; and
(iv) a data set comprising measurements of the level of hybridization or complex in samples from a plurality of healthy subjects.

The biological sample used in a method described herein according to any embodiment comprises a nucleated cell and/or an extract thereof. Preferably, the sample is selected from the group consisting of whole blood, serum, plasma, peripheral blood mononuclear cells (PBMC), a buffy coat fraction, saliva, urine, a buccal cell and a skin cell.

As will be apparent to the skilled artisan based on the description herein, the size of a sample will depend upon the detection means used. For example, an assay, such as, for example, PCR may be performed using a sample comprising a single cell or an extract thereof, although greater numbers of cells are preferred. Alternative forms of nucleic acid detection may require significantly more cells than a single cell. Furthermore, protein-based assays require sufficient cells to provide sufficient protein for an antigen based assay.

In one example, the sample has been derived or isolated or obtained previously from the subject.

In one example, the method of the invention described herein according to any embodiment is performed using genomic DNA obtained from a sample from a subject, e.g., obtained from a blood sample from a subject. Alternatively, or in addition, the method described herein according to any embodiment is performed using mRNA or cDNA derived from the biological sample. Alternatively, or in addition, the method described herein according to any embodiment is performed using protein derived from the biological sample.

In one example, the method described herein according to any embodiment is performed as a part of a multi-analyte detection method to determine the predisposition of a subject to a neurodegenerative disease or to diagnose a neurodegenerative disease. For example, such a multi-analyte method detects two or more nucleic acid markers that are associated with a neurodegenerative disease, for example, two or more markers described herein according to any embodiment. Alternatively, or in addition, a multi-analyte method detects one or more nucleic acid markers associated with a neurodegenerative disease as described herein according to any embodiment and one or more other markers associated with a neurodegenerative disease. The combination of nucleic acid-based and protein-based detection methods is contemplated by the present invention.

In one example of the invention, the method described herein according to any embodiment additionally comprises determining an association between the marker and a neurodegenerative disease. Suitable methods for determining an association between a marker and a disease or disorder are known in the art.

The methods of the present invention are also useful for determining a subject that is a carrier of a marker that is associated with and/or linked to a neurodegenerative disease. Such an assay is useful, for example, for determining the likelihood, or susceptibility of a child of the subject being tested to develop a neurodegenerative disease.

The present inventors have also determined at least one marker that occurs in subjects suffering from Alzheimer's disease. Accordingly, the present invention also provides a method for diagnosing a particular form of a neurodegenerative disease or determining a predisposition of a subject to developing a particular form of a neurodegenerative disease or determining a risk of a subject developing a neurodegenerative disease. For example, the particular form of a neurodegenerative disease is Alzheimer's disease or FTLD or motor neuron disease. For example, the methods described herein according to any embodiment apply mutatis mutandis to diagnosing Alzheimer's disease or FTLD or motor neuron disease or determining the predisposition of a subject to developing Alzheimer's disease or FTLD or motor neuron disease or determining the risk of a subject developing Alzheimer's disease or FTLD or motor neuron disease.

In one example, a method as described herein according to any embodiment additionally comprises determining a neurodegenerative disease that a subject suffers from or is predisposed to or has an increased risk of developing. Such a determination is based on, for example, family history or a physiological assay or a neurological assay or a molecular assay.

The diagnostic method of the present invention is also useful in a method of treatment. For example, the present invention provides a method of treatment or prophylaxis of a neurodegenerative disease, said method comprising:
(i) performing a method described herein for diagnosing a neurodegenerative disease or a predisposition thereto; and
(ii) administering or recommending a therapeutic or prophylactic compound for the treatment of the neurodegenerative disease.

Alternatively, the present invention provides a method of treatment or prophylaxis of a neurodegenerative disease, said method comprising:

(i) obtaining results of a method described herein according to any embodiment indicating that a subject suffers from a neurodegenerative disease or has a predisposition to a neurodegenerative disease; and
(ii) administering or recommending a therapeutic or prophylactic compound for the treatment of the neurodegenerative disease

In one embodiment, the administration or recommendation of a therapeutic for the treatment of the neurodegenerative disease is based upon the diagnosis of the disease or the diagnosis of a predisposition to the disease.

The present invention also provides a method for predicting the response of a subject to treatment with a composition for the treatment or prophylaxis of a neurodegenerative disease, said method comprising detecting a marker within an OPRS-1 gene or an expression product thereof that is associated with response of a subject to treatment with a composition for the treatment or prophylaxis of a neurodegenerative disease, wherein detection of said marker is indicative of the response of the subject to treatment with said composition.

In one example, the method detects a marker associated with a subject that will respond to treatment. As used herein, the term “respond to treatment” shall be taken to mean that the symptoms of a neurodegenerative disease in a subject are reduced or ameliorated as a result of treatment with a therapeutic compound.

Alternatively, a marker is associated with a subject that will not respond to treatment. As will be apparent to the skilled artisan from the preceding paragraph, the term “will not respond to treatment” means that a neurodegenerative disease or one or more symptoms of a neurodegenerative disease in a subject are unlikely to be reduced or ameliorated as a result of treatment with a therapeutic compound. For example, in a significant proportion of the population carrying a marker as described herein according to any embodiment, treatment with a therapeutic compound will not result in therapeutic benefit to the subject in the treatment of a neurodegenerative disease or one or more symptoms thereof. Proceeding on this basis, the term “will not respond to treatment” may be used interchangeably with the term “is unlikely to respond to treatment”.

In one example, the present invention provides a nucleic acid comprising a sequence set forth in SEQ ID NO: 7, wherein the sequence comprises a thymine at a position corresponding to nucleotide position 1005 of SEQ ID NO: 7. Alternatively, or in addition the present invention provides a nucleic acid comprising a sequence set forth in SEQ ID NO: 5, wherein the sequence comprises an adenosine at a position corresponding to nucleotide position 80 of SEQ ID NO: 5 and/or a thymine at a position corresponding to position 85 of SEQ ID NO: 5 and/or an adenosine at a position corresponding to nucleotide position 626 of SEQ ID NO: 5. Alternatively, or in addition, the present invention provides a nucleic acid comprising a sequence set forth in SEQ ID NO: 8, wherein the sequence comprises a thymine at a position corresponding to nucleotide position 699 of SEQ ID NO: 8. Alternatively, or in addition, the present invention provides a nucleic acid comprising a sequence set forth in SEQ ID NO: 13, wherein the sequence comprises a an adenosine at a position corresponding to position 2080 of SEQ ID NO: 13 and/or a thymine at a position corresponding to position 2092 of SEQ ID NO: 13 and/or a thymine at a position corresponding to position 2583 of SEQ ID NO: 13 and/or a thymine at a position corresponding to position 4020 of SEQ ID NO: 13 and/or a thymine at a position corresponding to position 4191 of SEQ ID NO: 13 and/or an adenosine at a position corresponding to position 4187 of SEQ ID NO: 13 and/or an adenosine at a position corresponding to nucleotide position 2254 of SEQ ID NO: 13, and/or an adenosine at a position corresponding to nucleotide position 2255 of SEQ ID NO: 13, and/or an adenosine at a position corresponding to nucleotide position 2257 of SEQ ID NO: 13, and/or an adenosine at a position corresponding to nucleotide position 2792 of SEQ ID NO: 13 and/or a thymidine at nucleotide position 141 of SEQ ID NO: 5.

The present invention also provides an isolated nucleic acid, e.g., a probe or primer, capable of preferentially or specifically hybridizing to or annealing to a nucleic acid described in the previous paragraph. For example, the probe or primer comprises a sequence selected from the group consisting of:

(i) a sequence of at least about 15 to 20 nucleotides of SEQ ID NO: 7, wherein the sequence comprises a thymine at a position corresponding to nucleotide position 1005 of SEQ ID NO: 7;
(ii) a sequence of at least about 15 to 20 nucleotides of SEQ ID NO: 5, wherein the sequence comprises an adenosine at a position corresponding to nucleotide position 80 of SEQ ID NO: 5 and/or a thymine at a position corresponding to position 85 of SEQ ID NO: 5 and/or an adenosine at a position corresponding to nucleotide position 626 of SEQ ID NO: 5;
(iii) a sequence of at least about 15 to 20 nucleotides of SEQ ID NO: 8, wherein the sequence comprises a thymine at a position corresponding to nucleotide position 699 of SEQ ID NO: 8;
(iv) a sequence of at least about 15 to 20 nucleotides of SEQ ID NO: 13, wherein the sequence comprises an adenosine at a position corresponding to position 2080 of SEQ ID NO: 13 and/or a thymine at a position corresponding to position 2092 of SEQ ID NO: 13 and/or a thymine at a position corresponding to position 2583 of SEQ ID NO: 13 and/or a thymine at a position corresponding to position 4020 of SEQ ID NO: 13 and/or a thymine at a position corresponding to position 4191 of SEQ ID NO: 13 and/or an adenosine at a position corresponding to position 4187 of SEQ ID NO: 13 and/or an adenosine at a position corresponding to nucleotide position 2254 of SEQ ID NO: 13, and/or an adenosine at a position corresponding to nucleotide position 2255 of SEQ ID NO: 13, and/or an adenosine at a position corresponding to nucleotide position 2257 of SEQ ID NO: 13, and/or an adenosine at a position corresponding to nucleotide position 2792 of SEQ ID NO: 13, and/or a thymidine at nucleotide position 141 of SEQ ID NO: 5; and
(v) the complement of any one of (i) to (iv).

By “preferentially” means that the probe or primer is used under conditions under which a target polynucleotide hybridizes to the probe or primer at a level significantly above background. The background hybridization may occur because of other polynucleotides present, for example, in the cDNA or genomic DNA library being screening or other cDNA or gDNA in a sample being screened. Background implies a level of signal generated by interaction between the probe and a non-target nucleic acid which is less than 10 fold, preferably less than 100 fold as intense as the specific interaction observed with the target nucleic acid. The intensity of interaction are measured, for example, by radiolabeling the probe, e.g. with ³²P. Preferably, a probe or primer that preferentially anneals or hybridizes to a sequence described supra, hybridizes or anneals to the target sequence to a greater level or degree than it does to another sequence, e.g., an allelic variant of a sequence set forth in SEQ ID NO: 5, 7, 8 or 13.

By “specifically” is meant that a probe or primer hybridizes or anneals to a target sequence and does not detectably anneal or hybridize to another target sequence, e.g., an allelic variant of a sequence set forth in SEQ ID NO: 5, 7, 8 or 13.

The present invention also provides an isolated protein comprising a sequence set forth in SEQ ID NO: 6 wherein the sequence comprises a valine at a position corresponding to position 4 of SEQ ID NO: 6.

The present invention also provides an isolated antibody or antigen binding fragment thereof capable of preferentially or specifically binding to a polypeptide comprising a sequence set forth in SEQ ID NO: 6 wherein the sequence comprises a valine at a position corresponding to position 4 of SEQ ID NO: 6 or a serine at a position corresponding to position 23 of SEQ ID NO: 6. For example, the antibody or fragment thereof binds to an epitope of OPRS1 polypeptide comprising a sequence comprising at least about five consecutive amino acids of SEQ ID NO: 6 wherein the sequence comprises a valine at a position corresponding to position 4 of SEQ ID NO: 6 or a serine at a position corresponding to position 23 of SEQ ID NO: 6. The terms “preferentially” and “specifically” are to be given the same meaning mutatis mutandis in respect of antibodies as they are in respect of probes and primers.

Given the tight association of the human OPRS-1 gene to a neurodegenerative disease, and the provision of a plurality of markers in OPRS-1 associated with a neurodegenerative disease, the present invention further provides methods for identifying new markers in an OPRS-1 gene or expression product associated with a neurodegenerative disease. For example, the present invention provides a method for identifying a marker in an OPRS-1 gene or expression product that is associated with a neurodegenerative disease, said method comprising:
(i) identifying a polymorphism or allele or mutation within an OPRS-1 gene or expression product thereof;
(ii) analyzing a panel of subjects to determine those that suffer from a neurodegenerative disease, wherein not all members of the panel comprise the polymorphism or allele or mutation; and
(iii) determining the variation in the development of the neurodegenerative disease wherein said variation indicates that the polymorphism or allele or mutation is associated with the neurodegenerative disease or a subject's predisposition to the neurodegenerative disease.

The present invention also provides a method of identifying a marker associated with dementia comprising identifying a marker that is linked to chromosome position 9p21, e.g. 9p21.1-9p21.2 of the human genome, wherein said marker is present in an individual suffering from dementia and said marker is not present in a suitable control subject. For example, the method described supra comprising identifying a polymorphism or allele or mutation within an OPRS1 gene shall be taken to apply mutatis mutandis to identifying a polymorphism or allele or mutation linked to chromosome position 9p21 of the human genome.

### DEFINITIONS

This specification contains nucleotide and amino acid sequence information prepared using PatentIn Version 3.3, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:” followed by the sequence identifier (e.g. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenosine, C represents Cytosine, G represents Guanine, T represents thymine, Y represents a pyrimidine residue, R represents a purine residue, M represents Adenosine or Cytosine, K represents Guanine or Thymine, S represents Guanine or Cytosine, W represents Adenosine or Thymine, H represents a nucleotide other than Guanine, B represents a nucleotide other than Adenosine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Each embodiment described herein with respect to the diagnosis of dementia and/or determining the predisposition of a subject to dementia shall be taken to apply mutatis mutandis to the diagnosis of presenile dementia and/or determining the predisposition of a subject to presenile dementia.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

### BRIEF DESCRIPTION OF THE DRAWINGS

FIG. 1 is a pedigree diagram showing affection status and disease haplotype of the early onset dementia family 14. Squares indicate males and circles females; filled arrow indicates proband; black symbols, show individuals clinically diagnosed with dementia, either AD or FTLD; diagonal stripes, individuals diagnosed with MND; and combined black and diagonal stripes, individuals diagnosed with FTLD-MND. A diagonal line marks deceased subjects. Individual I:1, lived until his 80s, but was thought to have had some personality changes. Alleles in parentheses are inferred. X indicates upper and lower recombination breakpoints which define the minimal disease haplotype.

FIG. 2 is a DNA sequence electropherogram showing the sequence of nucleotide changes observed in subjects suffering from a neurodegenerative disease. Nucleotide changes are represented by the vertical arrows. A common polymorphism is indicated by the asterisk (*).

FIG. 3 is a graphical representation showing the level of expression of the luciferase gene in SK-N-MC cells or SK-N-SH cells when placed under control of either the G723T mutation (Australian mutation) or G719A mutation (Polish mutation).

FIG. 4A shows a copy of a photographic representation showing of electrophoresis of exon trap products on a 2% agarose gel. Exon trapping was performed in HEK293 cells (left hand panel) and SK-N-MC (right hand panel), transfected with the pSPL3 vector containing wild type OPRS1 sequence (wt), pSPL3 vector comprising OPRS1 mutation in IVS+31 (IVS+31) or pSPL3 vector comprising OPRS1 mutation at IVS+24 (IVS+24).

FIG. 4B is a graphical representation showing results of semi-quantitative analysis of exon trap products isolated from HEK-293 cells (left hand panel) and SKNMC cells (right-hand panel). Mean values±SD obtained from four separate transfections. Pairwise Student's t test comparisons were performed between the T and C allele exon trap products. Statistical significance is indicated (*=p<0.05).

FIG. 5 is a copy of a graphical representation showing the level of gamma secretase activity in cells expressing wild-type OPRS1 (pcDNA-FLAG-OPRS1 (wt)) and mutant OPRS1 (Ala4Val; pcDNA-FLAG-OPRS1 (Ala4Val)), in SKNMC cells (light grey bars) and SKNSH cells (dark grey bars).

FIG. 6 is a graphical representation showing age-dependent effect of disease status on OPRS1 expression. OPRS1 cDNA levels in lymphoblastoid cell lines were assessed by quantitative real-time PCR and were calculated relative to the housekeeping gene SDHA. Expression levels were plotted against age at sample donation for 5 patients (grey squares) and 10 controls (black triangles).

FIG. 7 is a graphical representation showing a correlation (r²=0.852, p=0.006) between OPRS1 transcript levels and the relative amount of TDP-43 protein in the cytoplasm as expressed as a ratio of TDP-43 in cytoplasmic versus nuclear fraction. Accordingly, increased OPRS1 expression as is observed in subjects suffering from neurodegenerative disease is correlated with increased cytoplasmic TDP-43 levels, another marker of neurodegenerative disease.

FIG. 8 is a graphical representation showing the level of TPD-43 localized to the nucleus of cells when various forms of OPRS1 are overexpressed. White bars represent results in SKNMC cells and shaded bars represent results from SKNSH cells. Overexpression (i.e., increased levels of OPRS1 as seen in some subjects suffering from neurodegenerative disease) results in increased the level of TDP-43 in the cytoplasm of cells, a marker of neurodegenerative disease.

### DETAILED DESCRIPTION OF THE PREFERRED EMBODIMENTS

### 1. Markers Associated with a Disease or Disorder

In one example of the present invention, a marker associated with or causative of a neurodegenerative disease is a nucleic acid marker. Preferably, the marker comprises or consists of a nucleotide sequence at least about 80% identical to at least about 20 contiguous nucleotides, more preferably at least about 30 contiguous nucleotides, of a sequence selected from the group consisting of:
(i) a sequence selected from the group consisting of SEQ ID NO: 1-5, 7, 8 and 13;
(ii) a sequence capable of encoding an amino acid sequence at least 80% homologous to the sequence set forth in SEQ ID NO: 6; and
(iii) a sequence complementary to a sequence set forth in (i) or (ii).

Such a nucleic acid marker may be or comprise, for example, a polymorphism, an insertion into an OPRS1 gene or transcript thereof, a deletion from an OPRS1 gene or transcript thereof, a transcript of an OPRS1 gene or a fragment thereof or an alternatively spliced transcript of an OPRS1 or a fragment thereof.

In one example of the invention a marker comprises a polymorphism or more preferably a mutation associated with or causative of alternative splicing of an OPRS1 mRNA.

In one example, the presence of a polymorphism or mutation associated with alternative splicing of an OPRS1 mRNA is correlated with modulated levels of alternatively spliced OPRS1 mRNA, e.g., increased levels of a mRNA lacking nucleic acid compared to SEQ ID NO: 5 and/or reduced levels of a mRNA comprising a sequence set forth in SEQ ID NO: 5. Preferably, the marker comprises a sequence comprising a thymidine at a position corresponding to nucleotide position 2583 of SEQ ID NO: 13 or an adenosine at a position corresponding to nucleotide position 2576 of SEQ ID NO: 13. In another example a marker associated with or causative of alternatively splicing in an OPRS1 expression produce, e.g., transcript, comprises an adenosine at a position corresponding to nucleotide position 2254 of SEQ ID NO: 13, or an adenosine at a position corresponding to nucleotide position 2255 of SEQ ID NO: 13, or an adenosine at a position corresponding to nucleotide position 2257 of SEQ ID NO: 13, or an adenosine at a position corresponding to nucleotide position 2792 of SEQ ID NO: 13. The level of a specific splice form of OPRS1 mRNA is increased or decreased when the polymorphism is present and is useful for detecting a marker associated with a neurodegenerative disease.

The present inventors have additionally shown association of a nucleotide variation in the OPRS1 gene that increases expression of OPRS1 and the development of a neurodegenerative disease. Accordingly, in another embodiment of the invention, the marker comprises a polymorphism or mutation that increases expression of an OPRS1 expression product compared to the level of expression of an OPRS1 expression product expressed from a gene that does not comprise the polymorphism or mutation. Preferably, the marker comprises a sequence comprising a thymidine at a position corresponding to nucleotide position 4191 of SEQ ID NO: 13 and/or an adenosine at a position corresponding to nucleotide position 4187 of SEQ ID NO: 13

In another example, the marker is in an OPRS1 polypeptide. Preferably, the marker comprises a sequence comprising a valine at a position corresponding to amino acid residue 4 of SEQ ID NO: 6.

In one embodiment, the method of the invention comprises detecting or determining the presence of a plurality of markers associated with a neurodegenerative disease.

### 2. Nucleic Acid Detection Methods

As will be apparent to the skilled artisan a probe or primer capable of specifically detecting a marker that is associated with or causative of a neurodegenerative disease is any probe or primer that is capable of specifically hybridizing to the region of the genome that comprises said marker, or an expression product thereof. Accordingly, a nucleic acid marker is preferably at least about 8 nucleotides in length (for example, for detection using a locked nucleic acid (LNA) probe). To provide more specific hybridization, a marker is preferably at least about 15 nucleotides in length or more preferably at least 20 to 30 nucleotides in length. Such markers are particularly amenable to detection by nucleic acid hybridization-based detection means assays, such as, for example any known format of PCR or ligase chain reaction.

In one embodiment, a preferred probe or primer comprises, consists of or is within a nucleic acid comprising a nucleotide sequence at least about 80% identical to at least 20 nucleotides of a sequence selected from the group consisting of:
(i) a sequence at least about 80% homologous to a sequence selected from the group consisting of SEQ ID NO: 1-5, 7, 8 and 13;
(ii) a sequence capable of encoding an amino acid sequence at least 80% homologous to the sequence set forth in SEQ ID NO: 6; and
(iii) a sequence complementary to a sequence set forth in (i) or (ii).

Generally, a method for detecting a nucleic acid marker comprises hybridizing an oligonucleotide to the marker linked to nucleic acid in a sample from a subject under moderate to high stringency conditions and detecting hybridization of the oligonucleotide using a detection means, such as for example, an amplification reaction or a hybridization reaction.

For the purposes of defining the level of stringency to be used in these diagnostic assays, a low stringency is defined herein as being a hybridization and/or a wash carried out in 6×SSC buffer, 0.1% (w/v) SDS at 28° C., or equivalent conditions. A moderate stringency is defined herein as being a hybridization and/or washing carried out in 2×SSC buffer, 0.1% (w/v) SDS at a temperature in the range 45° C. to 65° C., or equivalent conditions. A high stringency is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art.

Generally, the stringency is increased by reducing the concentration of SSC buffer, and/or increasing the concentration of SDS and/or increasing the temperature of the hybridization and/or wash. Those skilled in the art will be aware that the conditions for hybridization and/or wash may vary depending upon the nature of the hybridization matrix used to support the sample DNA, and/or the type of hybridization probe used.

In another embodiment, stringency is determined based upon the temperature at which a probe or primer dissociates from a target sequence (i.e., the probe or primers melting temperature or Tm). Such a temperature may be determined using, for example, an equation or by empirical means. Several methods for the determination of the Tm of a nucleic acid are known in the art. For example the Wallace Rule determines the G+C and the T+A concentrations in the oligonucleotide and uses this information to calculate a theoretical Tm (Wallace et al., Nucleic Acids Res. 6, 3543, 1979). Alternative methods, such as, for example, the nearest neighbour method are known in the art, and described, for example, in Howley, et al., J. Biol. Chem. 254, 4876, Santa Lucia, Proc. Natl. Acad. Sci. USA, 95: 1460-1465, 1995 or Bresslauer et al., Proc. Natl. Acad. Sci. USA, 83: 3746-3750, 1986. A temperature that is similar to (e.g., within 5° C. or within 10° C.) or equal to the proposed denaturing temperature of a probe or primer is considered to be high stringency. Medium stringency is to be considered to be within 10° C. to 20° C. or 10° C. to 15° C. of the calculated Tm of the probe or primer.

### 2.1 Probe/Primer Design and Production

As will be apparent to the skilled artisan, the specific probe or primer used in an assay of the present invention will depend upon the assay format used. Clearly, a probe or primer that is capable of preferentially or specifically hybridizing or annealing to or detecting the marker of interest is preferred. Methods for designing probes and/or primers for, for example, PCR or hybridization are known in the art and described, for example, in Dieffenbach and Dveksler (Eds) (In: PCR Primer: A Laboratory Manual, Cold Spring Harbor Laboratories, NY, 1995). Furthermore, several software packages are publicly available that design optimal probes and/or primers for a variety of assays, e.g. Primer 3 available from the Center for Genome Research, Cambridge, Mass., USA. Probes and/or primers useful for detection of a marker associated with a neurodegenerative disease are assessed to determine those that do not form hairpins, self-prime or form primer dimers (e.g. with another probe or primer used in a detection assay).

Furthermore, a probe or primer (or the sequence thereof) is assessed to determine the temperature at which it denatures from a target nucleic acid (i.e. the melting temperature of the probe or primer, or Tm). Methods of determining Tm are known in the art and described, for example, in Santa Lucia, Proc. Natl. Acad. Sci. USA, 95: 1460-1465, 1995 or Bresslauer et al., Proc. Natl. Acad. Sci. USA, 83: 3746-3750, 1986.

A primer or probe useful for detecting a SNP or mutation in an allele specific PCR assay or a ligase chain reaction assay is designed such that the 3′ terminal nucleotide hybridizes to the site of the SNP or mutation. The 3′ terminal nucleotide may be any of the nucleotides known to be present at the site of the SNP or mutation. When complementary nucleotides occur in the probe or primer and at the site of the polymorphism the 3′ end of the probe or primer hybridizes completely to the marker of interest and facilitates amplification, for example, PCR amplification or ligation to another nucleic acid. Accordingly, a probe or primer that completely hybridizes to the target nucleic acid produces a positive result in an assay.

In another embodiment, a primer useful for a primer extension reaction is designed such that it preferentially o specifically hybridizes to a region adjacent to a specific nucleotide of interest, e.g. a SNP or mutation.

Whilst the specific hybridization of a probe or primer may be estimated by determining the degree of homology of the probe or primer to any nucleic acid using software, such as, for example, BLAST, the specificity of a probe or primer can only be determined empirically using methods known in the art.

A locked nucleic acid (LNA) or protein-nucleic acid (PNA) probe or a molecular beacon useful, for example, for detection of a SNP or mutation or microsatellite by hybridization is at least about 8 to 12 nucleotides in length. Preferably, the nucleic acid, or derivative thereof, that hybridizes to the site of the SNP or mutation or microsatellite is positioned at approximately the centre of the probe, thereby facilitating selective hybridization and accurate detection.

Methods for producing/synthesizing a probe or primer of the present invention are known in the art. For example, oligonucleotide synthesis is described, in Gait (Ed) (In: Oligonucleotide Synthesis: A Practical Approach, IRL Press, Oxford, 1984). For example, a probe or primer may be obtained by biological synthesis (eg. by digestion of a nucleic acid with a restriction endonuclease) or by chemical synthesis. For short sequences (up to about 100 nucleotides) chemical synthesis is preferable.

For longer sequences standard replication methods employed in molecular biology are useful, such as, for example, the use of M13 for single stranded DNA as described by J. Messing (1983) Methods Enzymol, 101, 20-78.

Other methods for oligonucleotide synthesis include, for example, phosphotriester and phosphodiester methods (Narang, et al. Meth. Enzymol 68: 90, 1979) and synthesis on a support (Beaucage, et al Tetrahedron Letters 22: 1859-1862, 1981) as well as phosphoramidate technique, Caruthers, M. H., et al., “Methods in Enzymology,” Vol. 154, pp. 287-314 (1988), and others described in “Synthesis and Applications of DNA and RNA,” S. A. Narang, editor, Academic Press, New York, 1987, and the references contained therein.

LNA synthesis is described, for example, in Nielsen et al, J. Chem. Soc. Perkin Trans., 1:3423, 1997; Singh and Wengel, Chem. Commun. 1247, 1998. While, PNA synthesis is described, for example, in Egholm et al., Am. Chem. Soc., 114: 1895, 1992; Egholm et al., Nature, 365: 566, 1993; and Orum et al., Nucl. Acids Res., 21: 5332, 1993.

In one embodiment, the probe or primer comprises one or more detectable markers. For example, the probe or primer comprises a fluorescent label such as, for example, fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7, fluorescein (5-carboxyfluorescein-N-hydroxysuccinimide ester), rhodamine (5,6-tetramethyl rhodamine). The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm).

Alternatively, the probe or primer is labeled with, for example, a fluorescent semiconductor nanocrystal (as described, for example, in U.S. Pat. No. 6,306,610), a radiolabel or an enzyme (e.g. horseradish peroxidase (HRP), alkaline phosphatase (AP) or β-galactosidase).

Such detectable labels facilitate the detection of a probe or primer, for example, the hybridization of the probe or primer or an amplification product produced using the probe or primer. Methods for producing such a labeled probe or primer are known in the art. Furthermore, commercial sources for the production of a labeled probe or primer will be known to the skilled artisan, e.g., Sigma-Genosys, Sydney, Australia.

The present invention additionally contemplates the use a probe or primer as described herein in the manufacture of a diagnostic reagent for diagnosing or determining a predisposition to a neurodegenerative disease.

### 2.2 Detection Methods

Methods for detecting nucleic acids are known in the art and include for example, hybridization based assays, amplification based assays and restriction endonuclease based assays. For example, a change in the sequence of a region of the genome or an expression product thereof, such as, for example, an insertion, a deletion, a transversion, a transition, alternative splicing or a change in the preference of or occurrence of a splice form of a gene is detected using a method, such as, polymerase chain reaction (PCR) strand displacement amplification, ligase chain reaction, cycling probe technology or a DNA microarray chip amongst others.

Methods of PCR are known in the art and described, for example, in Dieffenbach (Ed) and Dveksler (Ed) (In: PCR Primer: A Laboratory Manual, Cold Spring Harbor Laboratories, NY, 1995). Generally, for PCR two non-complementary nucleic acid primer molecules comprising at least about 20 nucleotides in length, and more preferably at least 30 nucleotides in length are hybridized to different strands of a nucleic acid template molecule, and specific nucleic acid molecule copies of the template are amplified enzymatically. PCR products may be detected using electrophoresis and detection with a detectable marker that binds nucleic acids. Alternatively, one or more of the oligonucleotides are labeled with a detectable marker (e.g. a fluorophore) and the amplification product detected using, for example, a lightcycler (Perkin Elmer, Wellesley, Mass., USA). Clearly, the present invention also encompasses quantitative forms of PCR, such as, for example, Taqman assays.

Strand displacement amplification (SDA) utilizes oligonucleotides, a DNA polymerase and a restriction endonuclease to amplify a target sequence. The oligonucleotides are hybridized to a target nucleic acid and the polymerase used to produce a copy of this region. The duplexes of copied nucleic acid and target nucleic acid are then nicked with an endonuclease that specifically recognizes a sequence at the beginning of the copied nucleic acid. The DNA polymerase recognizes the nicked DNA and produces another copy of the target region at the same time displacing the previously generated nucleic acid. The advantage of SDA is that it occurs in an isothermal format, thereby facilitating high-throughput automated analysis.

Ligase chain reaction (described in EU 320,308 and U.S. Pat. No. 4,883,750) uses at least two oligonucleotides that bind to a target nucleic acid in such a way that they are adjacent. A ligase enzyme is then used to link the oligonucleotides. Using thermocycling the ligated oligonucleotides then become a target for further oligonucleotides. The ligated fragments are then detected, for example, using electrophoresis, or MALDI-TOF. Alternatively, or in addition, one or more of the probes is labeled with a detectable marker, thereby facilitating rapid detection.

Cycling Probe Technology uses chimeric synthetic probe that comprises DNA-RNA-DNA that is capable of hybridizing to a target sequence. Upon hybridization to a target sequence the RNA-DNA duplex formed is a target for RNase H thereby cleaving the probe. The cleaved probe is then detected using, for example, electrophoresis or MALDI-TOF.

In a preferred embodiment, a marker that is associated with or causative of a neurodegenerative disease occurs within a protein coding region of a genomic gene (e.g. an OPRS1 gene) and is detectable in mRNA encoded by that gene. For example, such a marker may be an alternate splice-form of a mRNA encoded by a genomic gene (e.g. a splice form not observed in a normal and/or healthy subject, or, alternatively, an increase or decrease in the level of a splice form in a subject that carries the marker). Such a marker may be detected using, for example, reverse-transcriptase PCR (RT-PCR), transcription mediated amplification (TMA) or nucleic acid sequence based amplification (NASBA), although any mRNA or cDNA based hybridization and/or amplification protocol is clearly amenable to the instant invention.

Methods of RT-PCR are known in the art and described, for example, in Dieffenbach (Ed) and Dveksler (Ed) (In: PCR Primer: A Laboratory Manual, Cold Spring Harbor Laboratories, NY, 1995).

Methods of TMA or self-sustained sequence replication (3SR) use two or more oligonucleotides that flank a target sequence, a RNA polymerase, RNase H and a reverse transcriptase. One oligonucleotide (that also comprises a RNA polymerase binding site) hybridizes to an RNA molecule that comprises the target sequence and the reverse transcriptase produces cDNA copy of this region. RNase H is used to digest the RNA in the RNA-DNA complex, and the second oligonucleotide used to produce a copy of the cDNA. The RNA polymerase is then used to produce a RNA copy of the cDNA, and the process repeated.

NASBA systems rely on the simultaneous activity of three enzymes (a reverse transcriptase, RNase H and RNA polymerase) to selectively amplify target mRNA sequences. The mRNA template is transcribed to cDNA by reverse transcription using an oligonucleotide that hybridizes to the target sequence and comprises a RNA polymerase binding site at its 5′ end. The template RNA is digested with RNase H and double stranded DNA is synthesized. The RNA polymerase then produces multiple RNA copies of the cDNA and the process is repeated.

Clearly, the hybridization to and/or amplification of a marker associated with a neurodegenerative disease using any of these methods is detectable using, for example, electrophoresis and/or mass spectrometry. In this regard, one or more of the probes/primers and/or one or more of the nucleotides used in an amplification reactions may be labeled with a detectable marker to facilitate rapid detection of a marker, for example, marker as described supra, e.g., a fluorescent label (e.g. Cy5 or Cy3) or a radioisotope (e.g. ³²P).

Alternatively, amplification of a nucleic acid may be continuously monitored using a melting curve analysis method, such as that described in, for example, U.S. Pat. No. 6,174,670.

In a one exemplified form of the invention, a marker associated with a neurodegenerative disease comprises a single nucleotide change. Methods of detecting single nucleotide changes are known in the art, and reviewed, for example, in Landegren et al, Genome Research 8: 769-776, 1998.

For example, a single nucleotide changes that introduces or alters a sequence that is a recognition sequence for a restriction endonuclease is detected by digesting DNA with the endonuclease and detecting the fragment of interest using, for example, Southern blotting (described in Ausubel et al (In: Current Protocols in Molecular Biology. Wiley Interscience, ISBN 047 150338, 1987) and Sambrook et al (In: Molecular Cloning: Molecular Cloning: A Laboratory Manual, Cold Spring Harbor Laboratories, New York, Third Edition 2001)). Alternatively, a nucleic acid amplification method described supra, is used to amplify the region surrounding the single nucleotide changes. The amplification product is then incubated with the endonuclease and any resulting fragments detected, for example, by electrophoresis, MALDI-TOF or PCR.

The direct analysis of the sequence of polymorphisms of the present invention can be accomplished using either the dideoxy chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

Alternatively, a single nucleotide change is detected using single stranded conformational polymorphism (SSCP) analysis. SSCP analysis relies upon the formation of secondary structures in nucleic acids and the sequence dependent nature of these secondary structures. In one form of this analysis an amplification method, such as, for example, a method described supra, is used to amplify a nucleic acid that comprises a single nucleotide change. The amplified nucleic acids are then denatured, cooled and analyzed using, for example, non-denaturing polyarcrylamide gel electrophoresis, mass spectrometry, or liquid chromatography (e.g. HPLC or dHPLC). Regions that comprise different sequences form different secondary structures, and as a consequence migrate at different rates through, for example, a gel and/or a charged field. Clearly, a detectable marker may be incorporated into a probe/primer useful in SSCP analysis to facilitate rapid marker detection.

Alternatively, any nucleotide changes are detected using, for example, mass spectrometry or capillary electrophoresis. For example, amplified products of a region of DNA comprising a single nucleotide change from a test sample are mixed with amplified products from a normal/healthy individual. The products are denatured and allowed to reanneal. Clearly those samples that comprise a different nucleotide at the position of the single nucleotide change will not completely anneal to a nucleic acid molecule from a normal/healthy individual thereby changing the charge and/or conformation of the nucleic acid, when compared to a completely annealed nucleic acid. Such incorrect base pairing is detectable using, for example, mass spectrometry.

Mass spectrometry is also useful for detecting the molecular weight of a short amplified product, wherein a nucleotide change causes a change in molecular weight of the nucleic acid molecule (such a method is described, for example, in U.S. Pat. No. 6,574,700).

Allele specific PCR (as described, for example, In Liu et al, Genome Research, 7: 389-398, 1997) is also useful for determining the presence of one or other allele of a single nucleotide change. An oligonucleotide is designed, in which the most 3′ base of the oligonucleotide hybridizes with the single nucleotide change. During a PCR reaction, if the 3′ end of the oligonucleotide does not hybridize to a target sequence, little or no PCR product is produced, indicating that a base other than that present in the oligonucleotide is present at the site of single nucleotide change in the sample. PCR products are then detected using, for example, gel or capillary electrophoresis or mass spectrometry.

Primer extension methods (described, for example, in Dieffenbach (Ed) and Dveksler (Ed) (In: PCR Primer: A Laboratory Manual, Cold Spring Harbor Laboratories, NY, 1995)) are also useful for the detection of a single nucleotide change. An oligonucleotide that hybridizes to the region of a nucleic acid adjacent to the single nucleotide change. This oligonucleotide is then used in a primer extension protocol with a polymerase and a free nucleotide diphosphate that corresponds to either or any of the possible bases that occur at the single nucleotide change. Preferably the nucleotide-diphosphate is labeled with a detectable marker (e.g. a fluorophore). Following primer extension, unbound labeled nucleotide diphosphates are removed, e.g. using size exclusion chromatography or electrophoresis, or hydrolyzed, using for example, alkaline phosphatase, and the incorporation of the labeled nucleotide into the oligonucleotide is detected, indicating the base that is present at the site of the single nucleotide change. Alternatively, or in addition, as exemplified herein primer extension products are detected using mass spectrometry (e.g. MALDI-TOF).

Clearly, the present invention extends to high-throughput forms primer extension analysis, such as, for example, minisequencing (Sy Vämen et al., Genomics 9: 341-342, 1995). In such a method, a probe or primer (or multiple probes or primers) are immobilized on a solid support (e.g. a glass slide). A biological sample comprising nucleic acid is then brought into direct contact with the probe/s or primer/s, and a primer extension protocol performed with each of the free nucleotide bases labeled with a different detectable marker. The nucleotide present at a single nucleotide change or a number of single nucleotide changes is then determined by determining the detectable marker bound to each probe and/or primer.

Fluorescently labeled locked nucleic acid (LNA) molecules or fluorescently labeled protein-nucleic acid (PNA) molecules are useful for the detection of SNPs (as described in Simeonov and Nikiforov, Nucleic Acids Research, 30(17): 1-5, 2002). LNA and PNA molecules bind, with high affinity, to nucleic acid, in particular, DNA. Fluorophores (in particular, rhodomine or hexachlorofluorescein) conjugated to the LNA or PNA probe fluoresce at a significantly greater level upon hybridization of the probe to target nucleic acid. However, the level of increase of fluorescence is not enhanced to the same level when even a single nucleotide mismatch occurs. Accordingly, the degree of fluorescence detected in a sample is indicative of the presence of a mismatch between the LNA or PNA probe and the target nucleic acid, such as, in the presence of a SNP. Preferably, fluorescently labeled LNA or PNA technology is used to detect a single base change in a nucleic acid that has been previously amplified using, for example, an amplification method described supra.

As will be apparent to the skilled artisan, LNA or PNA detection technology is amenable to a high-throughput detection of one or more markers immobilizing an LNA or PNA probe to a solid support, as described in Orum et al., Clin. Chem. 45: 1898-1905, 1999.

Similarly, Molecular Beacons are useful for detecting single nucleotide changes directly in a sample or in an amplified product (see, for example, Mhlang and Malmberg, Methods 25: 463-471, 2001). Molecular beacons are single stranded nucleic acid molecules with a stem-and-loop structure. The loop structure is complementary to the region surrounding the single nucleotide change of interest. The stem structure is formed by annealing two “arms,” complementary to each other, that are on either side of the probe (loop). A fluorescent moiety is bound to one arm and a quenching moiety to the other arm that suppresses any detectable fluorescence when the molecular beacon is not bound to a target sequence. Upon binding of the loop region to its target nucleic acid the arms are separated and fluorescence is detectable. However, even a single base mismatch significantly alters the level of fluorescence detected in a sample. Accordingly, the presence or absence of a particular base at the site of a single nucleotide change is determined by the level of fluorescence detected.

A single nucleotide change can also be identified by hybridization to nucleic acid arrays, an example of which is described in WO 95/11995. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

Clearly the present invention encompasses other methods of detecting a single nucleotide change that is within an OPRS1 gene and associated with a neurodegenerative disease, such as, for example, SNP microarrays (available from Affymetrix, or described, for example, in U.S. Pat. No. 6,468,743 or Hacia et al, Nature Genetics, 14: 441, 1996), Taqman assays (as described in Livak et al, Nature Genetics, 9: 341-342, 1995), solid phase minisequencing (as described in Syvämen et al, Genomics, 13: 1008-1017, 1992), minisequencing with FRET (as described in Chen and Kwok, Nucleic Acids Res. 25: 347-353, 1997) or pyrominisequencing (as reviewed in Landegren et al., Genome Res., 8(8): 769-776, 1998).

In a preferred embodiment, a single nucleotide change in an OPRS1 gene or an expression product thereof that is associated with a neurodegenerative disease is detected using a Taqman assay essentially as described by Corder et al., Science, 261: 921-923.

### 3. Protein Detection Methods

### 3.1 Ligands and Antibodies

It will be apparent to the skilled artisan based on the disclosure herein that the present invention also extends to detection of a marker in a polypeptide, e.g., a polypeptide encoded by an alternatively spliced OPRS1 mRNA or an OPRS1 polypeptide comprising a sequence comprising a valine at a position corresponding to amino acid residue 4 of SEQ ID NO: 6. Methods for detecting such polypeptides generally make use of a ligand or antibody that preferentially or specifically binds to the target polypeptide. As used herein the term “ligand” shall be taken in its broadest context to include any chemical compound, polynucleotide, peptide, protein, lipid, carbohydrate, small molecule, natural product, polymer, etc. that is capable of selectively binding, whether covalently or not, to one or more specific sites on an OPRS1 polypeptide. The ligand may bind to its target via any means including hydrophobic interactions, hydrogen bonding, electrostatic interactions, van der Waals interactions, pi stacking, covalent bonding, or magnetic interactions amongst others. It is particularly preferred that a ligand is able to specifically bind to a specific form of an OPRS1 polypeptide, e.g. an OPRS1 polypeptide that comprises a valine at a position corresponding amino acid position 4 of SEQ ID NO: 6.

As used herein, the term “antibody” refers to intact monoclonal or polyclonal antibodies, immunoglobulin (IgA, IgD, IgG, IgM, IgE) fractions, humanized antibodies, or recombinant single chain antibodies, as well as fragments thereof, such as, for example Fab, F(ab)2, and Fv fragments.

Antibodies are prepared by any of a variety of techniques known to those of ordinary skill in the art, and described, for example in, Harlow and Lane (In: Antibodies: A Laboratory Manual, Cold Spring Harbor Laboratory, 1988). In one such technique, an immunogen comprising the antigenic polypeptide is initially injected into any one of a wide variety of animals (e.g., mice, rats, rabbits, sheep, humans, dogs, pigs, chickens and goats). The immunogen is derived from a natural source, produced by recombinant expression means, or artificially generated, such as by chemical synthesis (e.g., BOC chemistry or FMOC chemistry). In one example, an epitope of OPRS-1 comprising a valine at a position corresponding to amino acid residue 4 of SEQ ID NO: 6 serves as the immunogen.

A peptide, polypeptide or protein is joined to a carrier protein, such as bovine serum albumin or keyhole limpet hemocyanin. The immunogen and optionally a carrier for the protein is injected into the animal host, preferably according to a predetermined schedule incorporating one or more booster immunizations, and blood collected from said the animals periodically. Optionally, the immunogen is injected in the presence of an adjuvant, such as, for example Freund's complete or incomplete adjuvant, lysolecithin and dinitrophenol to enhance the subject's immune response to the immunogen. Monoclonal or polyclonal antibodies specific for the polypeptide are then purified from blood isolated from an animal by, for example, affinity chromatography using the polypeptide coupled to a suitable solid support.

Monoclonal antibodies specific for the antigenic polypeptide of interest are prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines are produced, for example, from spleen cells obtained from an animal immunized as described supra. The spleen cells are immortalized by, for example, fusion with a myeloma cell fusion partner, preferably one that is syngenic with the immunized animal. A variety of fusion techniques are known in the art, for example, the spleen cells and myeloma cells are combined with a nonionic detergent or electrofused and then grown in a selective medium that supports the growth of hybrid cells, but not myeloma cells. A preferred selection technique uses HAT (hypoxanthine, aminopterin, and thymidine) selection. After a sufficient time, usually about 1 to 2 weeks, colonies of hybrids are observed. Single colonies are selected and growth media in which the cells have been grown is tested for the presence of an antibody having binding activity against the polypeptide (immunogen). Hybridomas having high reactivity and specificity are preferred.

Monoclonal antibodies are isolated from the supernatants of growing hybridoma colonies using methods such as, for example, affinity purification as described supra.

Various techniques are also known for enhancing antibody yield, such as injection of the hybridoma cell line into the peritoneal cavity of a suitable vertebrate host, such as a mouse. Monoclonal antibodies are then harvested from the ascites fluid or the blood of such an animal subject. Contaminants are removed from the antibodies by conventional techniques, such as chromatography, gel filtration, precipitation, and/or extraction. The marker associated with neurodegeneration of this invention may be used in the purification process in, for example, an affinity chromatography step.

It is preferable that an immunogen used in the production of an antibody is one which is sufficiently antigenic to stimulate the production of antibodies that will bind to the immunogen and is preferably, a high titer antibody. In one embodiment, an immunogen is an entire protein.

In another embodiment, an immunogen consists of a peptide representing a fragment of a polypeptide, for example a region of an OPRS1 polypeptide that is alternatively spliced or an epitope of OPRS-1 comprising a valine at a position corresponding to amino acid residue 4 of SEQ ID NO: 6. Preferably an antibody raised to such an immunogen also recognizes the full-length protein from which the immunogen was derived, such as, for example, in its native state or having native conformation.

Alternatively, or in addition, an antibody raised against a peptide immunogen recognizes the full-length protein from which the immunogen was derived when the protein is denatured. By “denatured” is meant that conformational epitopes of the protein are disrupted under conditions that retain linear B cell epitopes of the protein. As will be known to a skilled artisan linear epitopes and conformational epitopes may overlap.

Alternatively, a monoclonal antibody capable of binding to a form of an OPRS1 polypeptide or a fragment thereof is produced using a method such as, for example, a human B-cell hybridoma technique (Kozbar et al., Immunol. Today 4:72, 1983), a EBV-hybridoma technique to produce human monoclonal antibodies (Cole et al. Monoclonal Antibodies in Cancer Therapy, 1985 Allen R. Bliss, Inc., pages 77-96), or screening of combinatorial antibody libraries (Huse et al., Science 246:1275, 1989).

Such an antibody is then particularly useful in detecting the presence of a marker of a neurodegenerative disease.

The methods described supra are also suitable for production of an antibody or antibody binding fragment as described herein according to any embodiment.

### 3.2 Detection Methods

In one embodiment, the method of the invention detects the presence of a marker in a polypeptide, aid marker being associated or causative of with a neurodegenerative disease.

An amount, level or presence of a polypeptide is determined using any of a variety of techniques known to the skilled artisan such as, for example, a technique selected from the group consisting of, immunohistochemistry, immunofluorescence, an immunoblot, a Western blot, a dot blot, an enzyme linked immunosorbent assay (ELISA), radioimmunoassay (RIA), enzyme immunoassay, fluorescence resonance energy transfer (FRET), matrix-assisted laser desorption/ionization time of flight (MALDI-TOF), electrospray ionization (ESI), mass spectrometry (including tandem mass spectrometry, e.g. LC MS/MS), biosensor technology, evanescent fiber-optics technology or protein chip technology.

In one example, an assay used to determine the amount or level of a protein is a semi-quantitative assay. In another example, an assay used to determine the amount or level of a protein in a quantitative assay.

Preferably, an amount of antibody or ligand bound to a marker of a neurodegenerative disease in an OPRS1 polypeptide is determined using an immunoassay. Preferably, using an assay selected from the group consisting of, immunohistochemistry, immunofluorescence, enzyme linked immunosorbent assay (ELISA), fluorescence linked immunosorbent assay (FLISA) Western blotting, RIA, a biosensor assay, a protein chip assay, a mass spectrometry assay, a fluorescence resonance energy transfer assay and an immunostaining assay (e.g. immunofluorescence).

Standard solid-phase ELISA or FLISA formats are particularly useful in determining the concentration of a protein from a variety of samples.

In one form such an assay involves immobilizing a biological sample onto a solid matrix, such as, for example a polystyrene or polycarbonate microwell or dipstick, a membrane, or a glass support (e.g. a glass slide). An antibody that specifically binds to a marker of a neurodegenerative disease in an OPRS1 polypeptide is brought into direct contact with the immobilized biological sample, and forms a direct bond with any of its target protein present in said sample. This antibody is generally labeled with a detectable reporter molecule, such as for example, a fluorescent label (e.g. FITC or Texas Red) or a fluorescent semiconductor nanocrystal (as described in U.S. Pat. No. 6,306,610) in the case of a FLISA or an enzyme (e.g. horseradish peroxidase (HRP), alkaline phosphatase (AP) or β-galactosidase) in the case of an ELISA, or alternatively a suitably labeled secondary antibody is used that binds to the first antibody. Following washing to remove any unbound antibody, the label is detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal) in the case of an enzymatic label.

Such ELISA or FLISA based systems are suitable for quantification of the amount of a protein in a sample, by calibrating the detection system against known amounts of a protein standard to which the antibody binds, such as for example, an isolated and/or recombinant OPRS1 polypeptide or immunogenic fragment thereof or epitope thereof.

In another form, an ELISA comprises immobilizing an antibody or ligand that specifically binds a marker of a disease or disorder within an OPRS1 polypeptide on a solid matrix, such as, for example, a membrane, a polystyrene or polycarbonate microwell, a polystyrene or polycarbonate dipstick or a glass support. A sample is then brought into physical relation with said antibody, and said marker within an OPRS1 polypeptide is bound or ‘captured’. The bound protein is then detected using a labeled antibody. For example, if the marker is captured from a human sample, a labeled anti-human OPRS1 antibody that binds to an epitope that is distinct from the first (capture) antibody is used to detect the captured protein. Alternatively, a third labeled antibody can be used that binds the second (detecting) antibody.

It will be apparent to the skilled person that the assay formats described herein are amenable to high throughput formats, such as, for example automation of screening processes or a microarray format as described in Mendoza et al., Biotechniques 27(4): 778-788, 1999. Furthermore, variations of the above-described assay will be apparent to those skilled in the art, such as, for example, a competitive ELISA.

Alternatively, the presence of a marker of a disease or disorder within an OPRS1 polypeptide is detected using a radioimmunoassay (RIA). The basic principle of the assay is the use of a radiolabeled antibody or antigen to detect antibody-antigen interactions. An antibody or ligand that specifically binds to the marker within an OPRS1 polypeptide is bound to a solid support and a sample brought into direct contact with said antibody. To detect the level of bound antigen, an isolated and/or recombinant form of the antigen is radiolabeled and brought into contact with the same antibody. Following washing, the level of bound radioactivity is detected. As any antigen in the biological sample inhibits binding of the radiolabeled antigen the level of radioactivity detected is inversely proportional to the level of antigen in the sample. Such an assay may be quantitated by using a standard curve using increasing known concentrations of the isolated antigen.

As will be apparent to the skilled artisan, such an assay may be modified to use any reporter molecule, such as, for example, an enzyme or a fluorescent molecule, in place of a radioactive label.

In another embodiment, Western blotting is used to determine the level of a marker within an OPRS1 polypeptide in a sample. In such an assay protein from a sample is separated using sodium doedecyl sulphate polyacrylamide gel electrophoresis (SDS-PAGE) using techniques known in the art and described in, for example, Scopes (In: Protein Purification: Principles and Practice, Third Edition, Springer Verlag, 1994). Separated proteins are then transferred to a solid support, such as, for example, a membrane (e.g., a PVDF membrane), using methods known in the art, for example, electrotransfer. This membrane is then blocked and probed with a labeled antibody or ligand that specifically binds to a marker of a neurodegenerative disease within an OPRS1. Alternatively, a labeled secondary, or even tertiary, antibody or ligand is used to detect the binding of a specific primary antibody. The level of label is then determined using an assay appropriate for the label used. An appropriate assay will be apparent to the skilled artisan.

For example, the level or presence a marker of a disease or disorder within an OPRS1 polypeptide is determined using methods known in the art, such as, for example, densitometry. In one example, the intensity of a protein band or spot is normalized against the total amount of protein loaded on a SDS-PAGE gel using methods known in the art. Alternatively, the level of the marker detected is normalized against the level of a control/reference protein. Such control proteins are known in the art, and include, for example, actin, glyceraldehyde 3-phosphate dehydrogenase (GAPDH), β2 microglobulin, hydroxy-methylbilane synthase, hypoxanthine phosphoribosyl-transferase 1 (HPRT), ribosomal protein L13c, succinate dehydrogenase complex subunit A and TATA box binding protein (TBP).

In an alternative embodiment, a marker of a neurodegenerative disease within an OPRS1 polypeptide is detected within a cell, using methods known in the art, such as, for example, immunohistochemistry or immunofluorescence. For example, a cell or tissue section that is to be analyzed to determine the presence of a marker of a neurodegenerative disease within an OPRS1 polypeptide is fixed to stabilize and protect both the cell and the proteins contained within the cell. Preferably, the method of fixation does not disrupt or destroy the antigenicity of the marker, thus rendering it undetectable. Methods of fixing a cell are known in the art and include for example, treatment with paraformaldehyde, treatment with alcohol, treatment with acetone, treatment with methanol, treatment with Bouin's fixative and treatment with glutaraldehyde. Following fixation a cell is incubated with a ligand or antibody capable of binding the marker. The ligand or antibody is, for example, labeled with a detectable marker, such as, for example, a fluorescent label (e.g. FITC or Texas Red), a fluorescent semiconductor nanocrystal (as described in U.S. Pat. No. 6,306,610) or an enzyme (e.g. horseradish peroxidase (HRP)), alkaline phosphatase (AP) or β-galactosidase. Alternatively, a second labeled antibody that binds to the first antibody is used to detect the first antibody. Following washing to remove any unbound antibody, the level of the bound to said labeled antibody is detected using the relevant detection means. Means for detecting a fluorescent label will vary depending upon the type of label used and will be apparent to the skilled artisan. Such a method is also useful for detecting subcellular localization of a TDP-43 polypeptide.

Optionally, a method of detecting a marker of a neurodegenerative disease within an OPRS1 polypeptide using immunofluorescence or immunohistochemistry will comprise additional steps such as, for example, cell permeabilization (using, for example, n-octyl-βD-glucopyranoside, deoxycholate, a non-ionic detergent such as Triton X-100 NP-40, low concentrations of ionic detergents, such as, for example SDS or saponin) and/or antigen retrieval (using, for example, heat).

Methods using immunofluorescence are preferable, as they are quantitative or at least semi-quantitative. Methods of quantitating the degree of fluorescence of a stained cell are known in the art and described, for example, in Immunohistochemistry (Cuello, 1984 John Wiley and Sons, ASIN 0471900524).

Biosensor devices generally employ an electrode surface in combination with current or impedance measuring elements to be integrated into a device in combination with the assay substrate (such as that described in U.S. Pat. No. 5,567,301). An antibody/ligand that specifically binds to a marker of a neurodegenerative disease within an OPRS1 polypeptide is preferably incorporated onto the surface of a biosensor device and a biological sample contacted to said device. A change in the detected current or impedance by the biosensor device indicates protein binding to said antibody. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit. This permits the simultaneous detection of several proteins or peptides in a small amount of body fluids.

Evanescent biosensors are also preferred as they do not require the pretreatment of a biological sample prior to detection of a protein of interest. An evanescent biosensor generally relies upon light of a predetermined wavelength interacting with a fluorescent molecule, such as for example, a fluorescent antibody attached near the probe's surface, to emit fluorescence at a different wavelength upon binding of the target polypeptide to the antibody or ligand.

Micro- or nano-cantilever biosensors are also preferred as they do not require the use of a detectable label. A cantilever biosensor utilizes a ligand and/or antibody capable of specifically detecting the analyte of interest that is bound to the surface of a deflectable arm of a micro- or nano-cantilever. Upon binding of the analyte of interest (e.g. a marker within an OPRS1 polypeptide) the deflectable arm of the cantilever is deflected in a vertical direction (i.e. upwards or downwards). The change in the deflection of the deflectable arm is then detected by any of a variety of methods, such as, for example, atomic force microscopy, a change in oscillation of the deflectable arm or a change in pizoresistivity. Exemplary micro-cantilever sensors are described in USSN 20030010097.

To produce protein chips, the proteins, peptides, polypeptides, antibodies or ligands that are able to bind specific antibodies or proteins of interest are bound to a solid support such as for example glass, polycarbonate, polytetrafluoroethylene, polystyrene, silicon oxide, metal or silicon nitride. This immobilization is either direct (e.g. by covalent linkage, such as, for example, Schiff's base formation, disulfide linkage, or amide or urea bond formation) or indirect. Methods of generating a protein chip are known in the art and are described in for example U.S. Patent Application No. 20020136821, 20020192654, 20020102617 and U.S. Pat. No. 6,391,625. To bind a protein to a solid support it is often necessary to treat the solid support so as to create chemically reactive groups on the surface, such as, for example, with an aldehyde-containing silane reagent. Alternatively, an antibody or ligand may be captured on a microfabricated polyacrylamide gel pad and accelerated into the gel using microelectrophoresis as described in, Arenkov et al. Anal. Biochem. 278:123-131, 2000.

A protein chip may comprise only one protein, ligand or antibody, and be used to screen one or more patient samples for the presence of one polypeptide of interest. Such a chip may also be used to simultaneously screen an array of patient samples for a polypeptide of interest.

Preferably, a protein sample to be analyzed using a protein chip is attached to a reporter molecule, such as, for example, a fluorescent molecule, a radioactive molecule, an enzyme, or an antibody that is detectable using methods known in the art. Accordingly, by contacting a protein chip with a labeled sample and subsequent washing to remove any unbound proteins the presence of a bound protein is detected using methods known in the art, such as, for example, using a DNA microarray reader.

Alternatively, biomolecular interaction analysis-mass spectrometry (BIA-MS) is used to rapidly detect and characterize a protein present in complex biological samples at the low- to sub-fmole level (Nelson et al. Electrophoresis 21: 1155-1163, 2000). One technique useful in the analysis of a protein chip is surface enhanced laser desorption/ionization-time of flight-mass spectrometry (SELDI-TOF-MS) technology to characterize a protein bound to the protein chip. Alternatively, the protein chip is analyzed using ESI as described in U.S. Patent Application 20020139751.

As will be apparent from the preceding discussion, it is particularly preferred to employ a detection system that is antibody or ligand based as such assays are amenable to the detection of a marker of a neurodegenerative disease within an OPRS1 polypeptide. Immunoassay formats are even more particularly preferred.

### Detection of an Enhanced or Reduced Level of an OPRS1 Transcript

The present inventors have also shown that nucleotide changes, e.g., mutations in an OPRS1 gene are associated with increased expression or reduced expression of a transcript of the OPRS1 gene in a subject suffering from a neurodegenerative disease. Accordingly, in one embodiment, a marker that is associated with a disease or disorder is detected by detecting an enhanced or reduced level of an OPRS1 transcript in a sample from a subject, wherein said enhanced or reduced level of the OPRS1 transcript is indicative of a neurodegenerative disease and/or a predisposition to a neurodegenerative disease and/or an increased risk of a subject developing a neurodegenerative disease.

In one example, the method comprises detecting an enhanced or reduced level of a native OPRS1 transcript, e.g., comprising a sequence set forth in SEQ ID NO: 5 wherein the nucleotide at position 80 is a guanine and the nucleotide at position 85 is cytosine and the nucleotide at position 626 is cytosine. Alternatively, the method comprises detecting an enhanced level of an alternatively spliced OPRS1 transcript.

Methods for detecting a transcript of an OPRS1 gene are described supra and are to be taken to apply mutatis mutandis to the present embodiment of the invention. For example, the level of an OPRS1 transcript is determined by performing a process comprising hybridizing a nucleic acid probe that selectively hybridizes to an OPRS1 transcript to nucleic acid in a sample from a subject under moderate to high stringency hybridization conditions and detecting the hybridization using a detection means, wherein the level of hybridization of the probe to the sample nucleic acid is indicative of the level of the OPRS1 transcript in the sample.

In one embodiment, an enhanced or reduced level of an OPRS1 transcript is detected by performing a process comprising:

(i) determining the level of the OPRS1 transcript in a sample from a subject;
(ii) comparing the level at (i) to the level in a suitable control sample, wherein an enhanced or reduced level of the OPRS1 transcript at (i) compared to (ii) is indicative of a neurodegenerative disease and/or a predisposition to a neurodegenerative disease and/or an increased risk of developing a neurodegenerative disease. A suitable control sample is described herein.

### Detection of an Enhanced or Reduced Level of an OPRS1 Polypeptide

The present inventors have also demonstrated that the level of expression of an OPRS1 polypeptide is associated with development of a neurodegenerative disease.

Accordingly, in one example, a marker associated with a neurodegenerative disease is detected by detecting an enhanced or reduced level of an OPRS1 polypeptide in a sample from a subject, wherein said enhanced or reduced level of the OPRS1 polypeptide is indicative of a neurodegenerative disease and/or a predisposition to a neurodegenerative disease and/or an increased risk of developing a neurodegenerative disease.

In one example, the method comprises detecting an enhanced or reduced level of a native OPRS1 polypeptide, e.g., comprising a sequence set forth in SEQ ID NO: 6 wherein the amino acid at position 4 is an alanine. Alternatively, the method comprises detecting an enhanced level of an OPRS1 polypeptide encoded by an alternatively spliced OPRS1 transcript.

Methods for determining the level of expression of a polypeptide are described supra and are to be taken to apply mutatis mutandis to the present aspect of the invention. For example, the level of the OPRS1 polypeptide is detected by performing a process comprising contacting a biological sample from a subject with an antibody or ligand capable of preferentially or specifically binding to the OPRS1 polypeptide for a time and under conditions sufficient for an antibody/ligand or ligand-ligand complex to form and then detecting the complex wherein the level of the complex is indicative of the level of the OPRS1 polypeptide in the subject.

Preferably, a method for detecting or determining an enhanced or reduced level of an OPRS1 polypeptide in a sample comprises performing a process comprising:
(i) determining the level of the OPRS1 polypeptide in the sample;
(ii) comparing the level of OPRS1 polypeptide at (i) to the level of OPRS1 polypeptide in a suitable control sample,
   wherein an enhanced or reduced level of the OPRS1 polypeptide at (i) compared to (ii) is indicative of a neurodegenerative disease and/or a predisposition to a neurodegenerative disease and/or an increased risk of developing a neurodegenerative disease. A suitable control sample will be apparent to the skilled artisan and/or is described herein.

### Monitoring the Efficacy of Treatment

The methods described herein are also to be taken to apply mutatis mutandis to a method for monitoring the efficacy of treatment of a neurodegenerative disease.

In one embodiment, the present invention provides a method for monitoring the efficacy of treatment of a subject undergoing treatment for a neurodegenerative disease, said method comprising:
(i) determining the level of expression of an OPRS1 expression product in a sample from a subject suffering from a neurodegenerative disease and receiving treatment therefor; and
(ii) determining the level of expression of the OPRS1 expression product in a suitable control sample,
   wherein a similar level of expression of the OPRS1 expression product at (i) compared to (ii) indicates that the treatment is effective for the treatment of the disease or disorder.

In this respect, a suitable control sample is a sample from a normal and/or healthy subject and/or a database comprising information concerning the level of expression of the OPRS1 expression product in a plurality of normal and/or healthy subjects.

### Biological Samples

As embodiments of the present invention are based upon detection of a marker in genomic DNA any cell or sample that comprises genomic DNA is useful for determining a disease or disorder and/or a predisposition to a disease or disorder. Preferably, the cell or sample is derived from a human. Preferably, comprises a nucleated cell.

Preferred biological samples include, for example, whole blood, serum, plasma, peripheral blood mononuclear cells (PBMC), a buffy coat fraction, saliva, urine, a buccal cell, urine, fecal material, sweat or a skin cell.

In a preferred embodiment, a biological sample comprises a white blood cell, more preferably, a lymphocyte cell.

Furthermore, as OPRS1 is widely expressed, any cell or sample comprising a cell may be used to determine a subject's predisposition to a neurodegenerative disease or to diagnose the disease on the basis of detecting an OPRS1 expression product provided that the cell expresses OPRS1.

Alternatively, the biological sample is a cell isolated using a method selected from the group consisting of amniocentesis, chorionic villus sampling, fetal blood sampling (e.g. cordocentesis or percutaneous umbilical blood sampling) and other fetal tissue sampling (e.g. fetal skin biopsy). Such biological samples are useful for determining the predisposition of a developing embryo to a neurodegenerative disease.

As will be apparent to the skilled artisan, the size of a biological sample will depend upon the detection means used. For example, an assay, such as, for example, PCR or single nucleotide primer extension may be performed on a sample comprising a single cell, although greater numbers of cells are preferred. Alternative forms of nucleic acid detection may require significantly more cells than a single cell. Furthermore, protein-based assays require sufficient cells to provide sufficient protein for an antigen based assay.

Preferably, the biological sample has been derived or isolated or obtained previously from the subject. Accordingly, the present invention also provides an ex vivo method. In one embodiment, the method of the invention additionally comprises isolating, obtaining or providing the biological sample.

In one embodiment, the method is performed using an extract from a biological sample, such as, for example, genomic DNA, mRNA, cDNA or protein.

As the present invention also includes detection of a marker in a OPRS1 gene that is associated with a disease or disorder in a cell (e.g. using immunofluorescence), the term “biological sample” also includes samples that comprise a cell or a plurality of cells, whether processed for analysis or not.

As will be apparent from the preceding description, such an assay may require the use of a suitable control, e.g. a normal individual or a typical population, e.g., for quantification.

As used herein, the term “normal individual” shall be taken to mean that the subject is selected on the basis that they do not comprise or express a marker that comprises, consists of or is within an OPRS1 gene or expression product thereof and that is associated with a neurodegenerative disease, nor do they suffer from a neurodegenerative disease.

For example, the normal subject has not been diagnosed with any form of neurodegenerative disease, using, for example, clinical analysis. For example, a subject may be tested for a neurodegenerative disease using a neuropsychological test (e.g. a Wechsler Adult Intelligence Scale test, MDRS or GDS), an EEG, a CAT scan or a MRI scan.

Alternatively, or in addition, a suitable control sample is a control data set comprising measurements of the marker being assayed for a typical population of subjects known not to suffer from a neurodegenerative disease. Preferably the subject is not at risk of developing such a disease, and, in particular, the subject does not have a family history of the disease.

In the present context, the term “typical population” with respect to subjects known not to suffer from a disease or disorder and/or comprise or express a marker of a neurodegenerative disease shall be taken to refer to a population or sample of subjects tested using, for example, known methods for diagnosing the neurodegenerative disease and determined not to suffer from the disease and/or tested to determine the presence or absence of a marker of the disease, wherein said subjects are representative of the spectrum of normal and/or healthy subjects or subjects known not to suffer from the disease.

Given that many diseases are quantitative traits, a subject may suffer from the disease and not comprise or express a marker of the disease described herein. Alternatively, a subject may not suffer from the disease, yet comprise or express a marker of as described herein. However, a suitable control sample for the instant invention is a sample derived from a subject that does not suffer from the disease and does not comprise or express a marker of the disease (e.g., as described herein).

In one embodiment, a reference sample is not included in an assay. Instead, a suitable reference sample is derived from an established data set previously generated from a typical population. Data derived from processing, analyzing and/or assaying a test sample is then compared to data obtained for the sample population.

Data obtained from a sufficiently large number of reference samples so as to be representative of a population allows the generation of a data set for determining the average level of a particular parameter. Accordingly, the amount of an expression product that is diagnostic of a neurodegenerative disease or a predisposition to a neurodegenerative disease can be determined for any population of individuals, and for any sample derived from said individual, for subsequent comparison to levels of the expression product determined for a sample being assayed. Where such normalized data sets are relied upon, internal controls are preferably included in each assay conducted to control for variation.

Methods for Determining a Marker Associated with a Disease or Disorder

In one embodiment, the method of the invention additionally comprises determining an association between a marker in an OPRS1 gene or expression product and a neurodegenerative disease.

Furthermore, given the tight association of the human OPRS1 gene to a neurodegenerative disease, and the provision of several markers associated with a neurodegenerative disease, the present invention further provides methods for identifying new markers for a neurodegenerative disease.

Accordingly, the present invention additionally provides a method for identifying a marker that is associated with a neurodegenerative disease, said method comprising:

(i) identifying a polymorphism or allele or mutation within an OPRS1 gene or an expression product thereof;
(ii) analyzing a panel of subjects to determine those that suffer from a neurodegenerative disease, wherein not all members of the panel comprise the polymorphism or allele or mutation; and
(iii) determining the variation in the development of the neurodegenerative disease wherein said variation indicates that the polymorphism or allele or mutation is associated with a subject's predisposition to the neurodegenerative disease.

Methods for determining the association between a marker and a disease, disorder and/or a phenotype are known in the art and reviewed, for example, in King (Ed) Rotter (Ed) and Motulski (Ed), The Genetic Basis of Common Disease, Oxford University Press, 2nd Edition, ISBN 0195125827, and Miller and Cronin (Eds), Genetic Polymorphisms and Susceptibility to Disease, Taylor and Francis, 1st Edition, ISBN 0748408223.

Generally, determining an association between a marker (e.g. a polymorphism and/or allele and/or a splice form and/or a mutation) and a disease, disorder or phenotype involves comparing the frequency of a polymorphism, allele, splice form or mutation at a specific locus between a sample of unrelated affected individuals (i.e., they comprise the phenotype of interest and/or suffer from the disease/disorder of interest) and an appropriate control that is representative of the allelic distribution in the normal population.

Several methods are useful for determining an association between a marker and a disease, disorder and/or phenotype of interest. However, any such study should consider several parameters to avoid difficulties, such as, for example, population stratification, that may produce false positive results.

Population stratification occurs when there are multiple subgroups with different allele frequencies present within a population. The different underlying allele frequencies in the sampled subgroups may be independent of the disease, disorder and/or phenotype within each group, and, as a consequence, may produce erroneous conclusions of linkage disequilibrium or association.

Generally, problems of population stratification are avoided by using appropriate control samples. For example, case-comparison based design may be used in which a comparison between a group of unrelated probands with the disease, disorder and/or phenotype and a group of control (comparison) individuals who are unrelated to each other or to the probands, but who have been matched to the proband group on relevant variable (other than affection status) that may influence genotype (e.g. sex, ethnicity and/or age).

Alternatively, controls are screened to exclude those subjects that have a personal history of the disease, disorder and/or phenotype of interest (and/or a family history of the disease, disorder and/or phenotype of interest). Such a “supernormal” control group is representative of the allele distribution of individuals unaffected by a disease, disorder and/or phenotype of interest.

Alternatively, a family-based association method may be used, in which non-transmitted alleles of the parents of a singly, ascertained proband are used as a random sample of alleles from which the proband was sampled. Such non-transmitted alleles are used to construct a matched control sample.

One extension of a family-based association method, the transmission disequilibrium test (TDT) uses a McNemar statistic to test for excess transmission of a marker allele to affected individuals above that expected by chance (Spielman et al., Am. J. Hum. Genet., 52: 506-516, 1993). Essentially, TDT considers parents who are heterozygous for an allele and/or polymorphism and/or splice variant associated with a disease, disorder or phenotype and evaluates the frequency with which the allele and/or polymorphism and/or splice variant or its alternate is transmitted to affected offspring. By only studying heterozygous parental genotypes TDT provides a test of association between linked loci and, as a consequence, avoids false associations between unlinked loci in the presence of population stratification.

The TDT method has been further refined to account for, for example multiallelic markers (Sham and Curtis Ann. Hum. Genet., 59: 323-326, 1995), multiple siblings in a family (Spielman and Ewens Am. J. Hum. Genet., 62:450-458, 1998), missing parental data (Curtis, Ann. Hum. Genet., 61: 319-333, 1997) and quantitative traits (Allison, Am. J. Hum. Genet., 60: 676-690, 1997 and Martin et al., Am. J. Hum. Genet., 67: 146-154, 2000).

In general, analysis of association is a test to detect non-random distribution of one or more alleles and/or polymorphisms and/or splice variants within subjects affected by a disease/disorder and/or phenotype of interest. The comparison between the test population and a suitable control population is made under the null hypothesis assumption that the locus to which the alleles and/or polymorphisms are linked has no influence on phenotype, and from this a nominal p-value is produced. For analysis of a biallelic polymorphism or mutation (e.g. a SNP) using a case control study, a chi-square analysis (or equivalent test) of a 2×2 contingency table (for analysis of alleles) or a 3×2 contingency table (for analysis of genotypes) is used.

For analysis using a family-based association study, marker data from members of the family of each proband are used to estimate the expected null distributions and an appropriate statistical test performed that compares observed data with that expected under the null hypothesis.

Another method useful in the analysis of association of a marker with a disease, disorder and/or phenotype is the genomic control method (Devlin and Roeder, Biometrics, 55: 997-1004, 1999). For a case-control analysis of candidate allele/polymorphism the genetic control method computes chi-square test statistics for both null and candidate loci. The variability and/or magnitude of the test statistics observed for the null loci are increased if population stratification and/or unmeasured genetic relationships among the subjects exist. This data is then used to derive a multiplier that is used to adjust the critical value for significance test for candidate loci. In this manner, genetic control permits analysis of stratified case-control data without an increased rate of false positives.

A structured association approach (Pritchard et al, Am. J. Hum. Genet., 67: 170-181, 2000) uses marker loci unlinked to a candidate marker to infer subpopulation membership. Latent class analysis is used to control for the effect of population substructure. Essentially, null loci are used to estimate the number of subpopulations and the probability of a subject's membership to each subpopulation. This method is then capable of accounting for a change in allele/polymorphism frequency as a result of population substructure.

Alternatively, or in addition, should a particular gene or gene product be likely to be involved in a disease, disorder or phenotype of interest a Bayesian statistical approach may be used to determine the significance of an association between an allele and/or polymorphism of that gene and the disease, disorder or phenotype of interest. Such an approach takes account of the prior probability that the locus under examination is involved in the disease, disorder or phenotype of interest (e.g., Morris et al., Am. J. Hum. Genet., 67:155-169, 2001).

Publicly available software is used to determine an association between an allele and/or polymorphism and/or a splice form and a disease or disorder or a predisposition to a disease or disorder. Such software include, for example, the following:
(i) Analysis of Complex Traits (ACT), which includes methods for multivariate analysis of complex traits. ACT is based on the research reported in Amos, et al., Ann. Hum. Genet. 60:143-160, 1996 and Amos, Am. J. Hum. Genet., 54:535-543, 1994;
(ii) ADMIXMAP, a general-purpose program for modeling admixture using marker genotypes and trait data of individuals from an admixed population, useful for estimate individual and population level admixture, test for a relationship between disease risk and individual admixture in case-control, cross-sectional or cohort studies, localize genes underlying ethnic differences in disease risk by admixture mapping and control for population structure (variation in individual admixture) in genetic association studies so as to eliminate associations with unlinked genes;
(iii) ANALYZE, an accessory program for the LINKAGE program that facilitates both parametric and non-parametric tests for association;
(iii) BAMA (Bayesian analysis of multilocus association), useful for selecting a trait-associated subset of markers among many candidates; and
(iv) CLUMP, a Monte Carlo method for assessing significance of a case-control association study with multi-allelic marker;
(v) ET-TDT (evolutionary tree—transmission disequilibrium test) and ETTDT (extended transmission disequilibrium test), extensions of the TDT test; and
(vi) FBAT (family based association test), useful for testing for association/linkage between disease phenotypes and haplotypes by utilizing family-based controls

Preferably, a marker that is determined using any of the methods described supra is within an OPRS1 gene or expression product and is associated with a neurodegenerative disease.

The present invention is described further in the following non-limiting examples:

### Example 1

### Identification of a FTLD Locus on Chromosome 9

### 1.1 Neuropathology

The brains of patients III:2, III:3 and III:12 and the spinal cord of patient III:12 were obtained at the time of autopsy with consent. The entire brain for III:3, the left hemi-brain and spinal cord for III:12, and the left hemi-brain for III:2 were fixed in 15% buffered formalin for at least 2 weeks. For each case routine neuropathological assessment, including immunohistochemistry screening, was performed at the time of autopsy and reviewed and standardized for the present study. After routine macroscopic assessment of the fixed tissue, blocks were excised from the frontal, parietal, occipital and limbic cortices, hippocampus, basal ganglia, thalamus, hypothalamus, midbrain, pons, medulla oblongata and cerebellum. For patient III:12, blocks of various spinal cord segments were also excised. All tissue blocks were paraffin-embedded, cut at 7 microns on a microtome, and mounted onto salinized slides. Routine stains included haemotoxylin and eosin (H & E), myelin and silver (Bielschowsky) stains. For all cases, retrospective review of standardized immunoperoxidase slides using antibodies for tau (MN1020, PIERCE, USA, diluted 1:10,000/cresyl violet), ubiquitin (Z0458, DAKO, Denmark, diluted 1:200/cresyl violet), Aβ and α-synuclein (610787, Pharmigen, USA, diluted 1:200/cresyl violet) were undertaken as previously described (Halliday et al., Acta Neuropthol., 90: 68-75 1995). To determine final diagnoses all cases were screened using current diagnostic criteria for AD (Hyman and Trojanowski, J. Neuropathol. Exp. Neurol., 56: 1095-1097 1997), dementia with Lewy bodies (McKeith et al., Neurology, 65: 1863-1872 2005), FTLD (Cairns et al., Acta Neuropathol, 114: 5-22 2007), MND (Brooks, J. Neurol. Sci., 124 Suppl: 96-107, 1994), and other neurodegenerative syndromes including corticobasal degeneration (Dickson et al., J. Neuropthol. Exp. Neurol., 61: 935-946, 2002), progressive supranuclear palsy (Hauw et al., Neurology, 44: 2015-2019, 1994) and vascular dementia (Nagata et al., J. Neurol. Sci., 257: 44-48, 2007).

### 1.2 Assessment of TDP-43-Immunopositive Inclusions

Paraffin-embedded 7 micron sections of the superior frontal cortex and hippocampus, as well as spinal cord sections for individual III:12, were obtained from the South Australian Brain Bank. TDP-43 protein was visualized following microwave antigen retrieval (sections were boiled for 3 min in 0.2M citrate buffer, pH 6.0) using commercially available antibody (BC001487, PTG, USA, diluted 1:500), peroxidase visualization and counterstaining with 0.5% cresyl violet. The location of the abnormal TDP-43-immunoreactive protein deposits within layer II neurons of the frontal cortex and hippocampal granule cells was identified as either cytoplasmic, intranuclear or neuritic. These features were used to classify the cases into histological subtypes according Sampathu et al. Am. J. Pathol. 169: 1343-1352, 2006. Similar immunohistochemical methods were used to identify α-internexin-positive inclusions using commercially available antibody (32-3600, ZYMED Laboratories, USA, diluted 1:50) and counterstaining with 0.5% cresyl violet.

### 1.3 Genetic Studies

After written informed consent was obtained, blood was collected from 16 family members (7 of whom are affected) and DNA extracted. Direct DNA sequencing of the coding regions and 50 base pairs of flanking intronic sequences was performed to screen the known dementia and MND genes (APP, PSEN1, PSEN2, MAPT, VCP, PGRN, IFT74, CHMP2B and SOD1).

Simulation analysis using SIMLINK version 4.12, was carried out to evaluate the power of the pedigree to detect linkage (Ploughman and Boehnke, Am. J. Hum. Genet., 44: 543-551, 1989). The estimated maximum logarithm-of-odds (LOD) score was based on 1000 simulations for a single marker with three alleles and equal allele frequencies where all clinical variants were assumed affected.

A 10 cM genome-wide scan was performed on DNA from 16 individuals by the Australian Genome Research Facility (AGRF) with microsatellite markers from the ABI-400 set (ABI Prism Linkage Mapping Set, version 2.5, MD-10). Parametric pair-wise and multipoint LOD scores were calculated using the MLINK and LINKMAP computer programs in the LINKAGE 5.2 package. Autosomal dominant inheritance was assumed with age dependent penetrance, a phenocopy rate of 0.005, a disease gene frequency of 0.001 and equal allele frequencies. Seven liability classes were established based on pedigree data with 1% penetrance—age <25 years, 8%—between 26 and 34 years, 22%—between 35 and 44 years, 46%—between 45 and 54 years, 71%—between 55 and 64 years, 91%—between 65 and 74 years, and 95%—age >75 years. Individuals were assigned a liability class based on age-of-onset for affected cases and age at last consultation for asymptomatic cases. High-resolution fine mapping was performed using microsatellite markers with an average heterozygosity of 0.79 and spaced no further apart than 2 cM. Markers were selected from the Marshfield Medical Research Foundation genetic framework map.

Primers were fluorescently labeled with FAM and PCR was carried out according to standard protocols. The amplified products were run on the Applied Biosystems 3730 DNA Analyser at the Ramaciotti Centre, University of New South Wales and analyzed using ABI software (Genotyper 2.5 and GeneScan 3.1, Applied Biosystems). Markers with a high rate of discrepancy were removed from the analysis. Haplotypes were constructed using Merlin (Version 2.01), double checked manually, and displayed using HaploPainter V.029.5 (Thiele and Nurnberg, 2005). The haplotype of individual III:5 was inferred from their spouse and offspring.

### 1.4 Results

### Clinical and Neuropathological Examinations of Affected Members

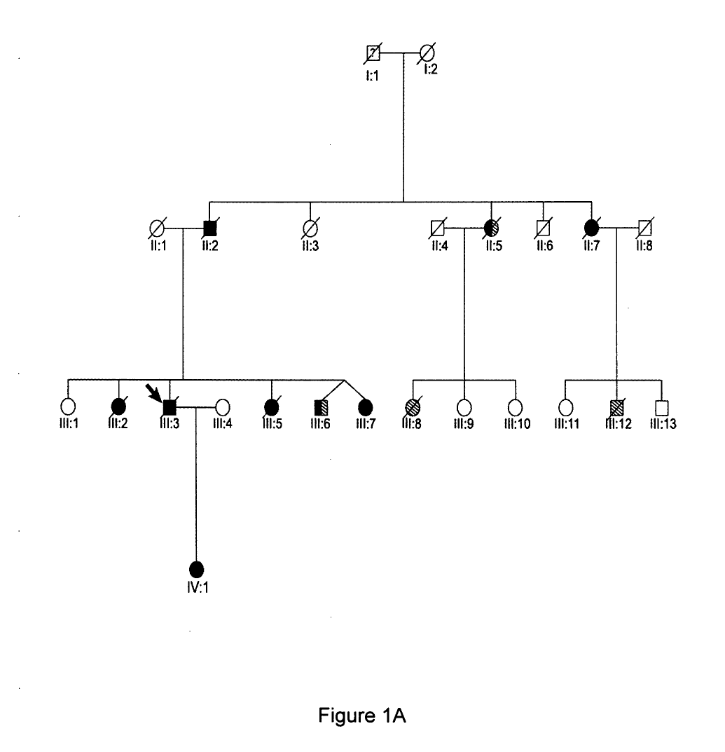

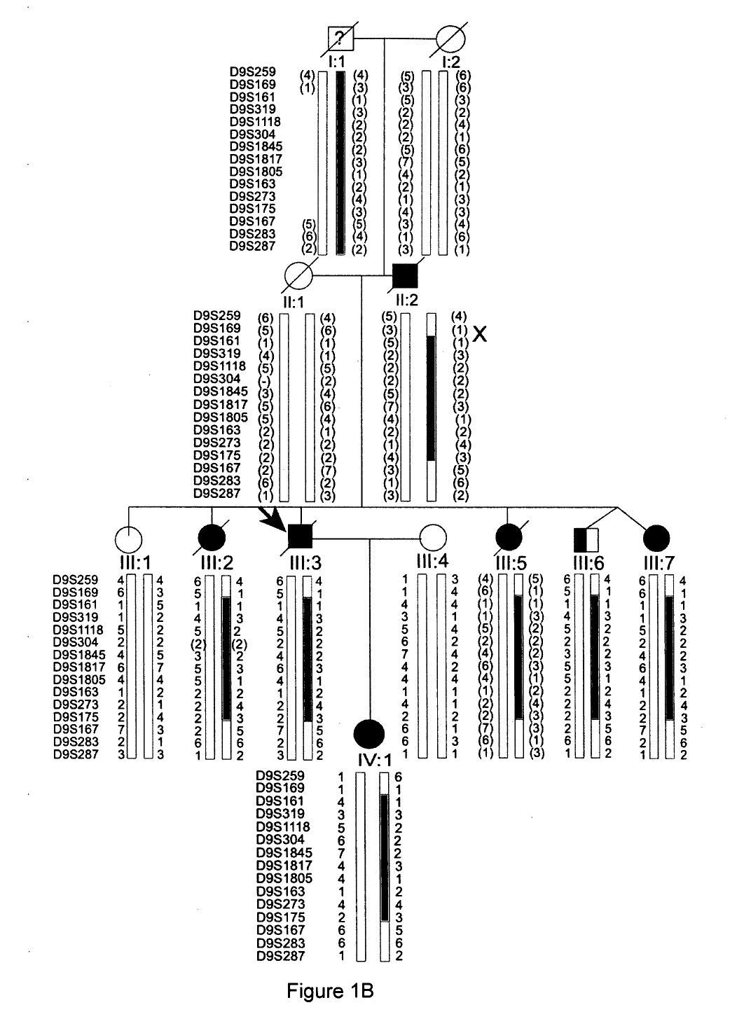

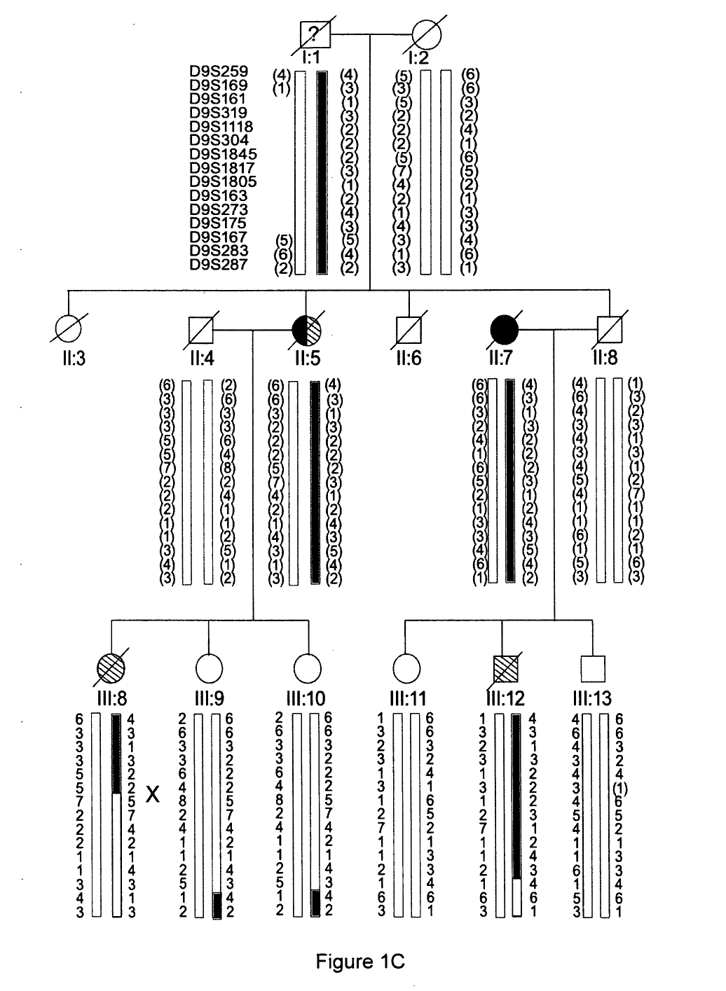

Australian family of Anglo-Celtic origin where eleven family members were affected with FTLD-MND was identified (FIG. 1). Over three generations, five family members (II:2, III:3, III:5, III:7, IV:1) presented with symptoms consistent with the behavioral variant of FTLD, with histopathologic confirmation in one (III:3). Another two family members (III:8, III:12) presented with progressive bulbar and limb weakness consistent with MND, with histopathologic confirmation in one (III:12). Two family members presented with a combination of FTLD and MND features (II:5, III:6). One family member presented with an amnestic picture clinically but was also found to have positive TDP-43 immunostaining on autopsy (III:2). One other family member presented with early-onset dementia (II:7) and had a son with MND (III:12). Of the eleven affected family members, two also developed paranoid delusions in their middle age, at the beginning of their illness (III:6 and III:8). Age of onset ranged from 43-68 and age of death 46-75.

### Mutation Screen of Pedigree Members

DNA from the proband (III:3), III:6, III:12 and III:1 was subjected to DNA sequence analysis of the coding regions and flanking intronic sequences for the known dementia and MND genes. No mutations were detected in the known dementia genes, namely APP, PSEN1, PSEN2, MAPT, PGRN, VCP, CHMP2B or the IFT74 gene. SOD1 was also negative for mutations in individuals III:8 and III:12.

### Linkage of Causative Locus to Chromosome 9p

The theoretical maximal two-point LOD score that could be obtained from the family 14 pedigree (FIG. 1) is 3.17 according to the power calculations using SIMLINK, with an average expected LOD score of 1.23. A genome-wide linkage analysis using the 400 ABI Linkage Mapping Set II markers was undertaken on 16 pedigree members, some of whom are not included in the pedigree diagram for ethical reasons. Seven individuals were classed as affected and one was classified as unknown as she had psychosis, a possible FTLD prodromal feature.

All available microsatellite data for 22 autosomes was uploaded into the Vincent database (Garvan Institute of Medical Research) and files were generated to enable statistical analysis using the LINKAGE package (MLINK and LINKMAP). Linkage analysis was carried out where a single genetic locus was considered causal for all clinical variants.

Over the entire genome, the only region with a two-point LOD score greater than the established cut-off of 2.0 for suggestive linkage was located on chromosome 9. MarkerD9S161 (9p21.3) gave a maximum LOD score of 2.57. Three adjacent markers also had positive LOD scores with the closest marker D9S1817 having a maximum LOD score of 0.99. The highest LOD score on a chromosome other than 9 was 1.40 on 3p14.3. Otherwise all other LOD scores were all consistently negative or non-significant and were used to exclude other reported MND linked loci, namely 2p13, 15q15-q22, 18q, 16q, and 20q13. These results indicate that the pedigree may be linked to the chromosome 9p FTLD-MND locus.

The candidate chromosome 9p region was subjected to high resolution fine mapping with 8 additional markers (D9S259, D9S169, D9S319, D9S1118, D9S304, D9S1845, D9S1805, D9S163) surrounding D9S161 and D9S1817 and the data was re-analyzed using MLINK. This resulted in a significant two-point LOD score of 3.25 at marker D9S319. To confirm this linkage and to identify the 95% confidence interval, parametric multipoint linkage analysis was carried out with markers D9S259, D9S169, D9S161, D9S319, D9S1118, D9S1845, D9S1817, D9S163, D9S273, D9S175 and D9S167. A peak multipoint LOD score of 3.79 at marker D9S319 was attained. The 95% confidence interval, as defined by the Zmax-1 score, identified a 12 cM region with markers D9S169 and D9S273 bordering this region.

To further evaluate the reliability of the detected linkage, and to determine recombination breakpoints, haplotypes were constructed using Merlin (FIG. 1). Recombination breakpoints were defined by two affected individuals. The telomeric boundary was marked by a recombination event seen in individual II:2 between markers D9S169 and D9S161. The centromeric boundary was defined by a single cross-over in individual III:8. However, the exact recombination breakpoint could not be determined as markers D9S1118 and D9S304 are both homozygous for the ‘2’ allele and could not be excluded from the disease haplotype. A cross-over was detected between markers D9S304 and D9S1845. All affected individuals share an identical haplotype consisting of 4 consecutive markers (D9S161-D9S319-D9S1118-D9S304) spanning a 9.6 cM region corresponding to a physical distance of 5.9 Mb.

The minimal disease region described supra was defined by a recombination event in individual II:2 (between markers D9S169 and D9S161) and a centromeric recombination in individual III:8 (between markers D9S304 and D9S1845). This region contains 14 known genes as listed by the UCSC Bioinformatics page [http://genome.ucsc.edu], consisting of C9orf11 (ACR formation associated factor), MOBKL2B, IFNK, c9orf72, LINGO2, ACO1, DDX58, TOPORS, NDUFB6, TAF1L, APTX, DNAJA1, SMU1, and B4GALT1. The coding and non-coding exonic sequence and flanking intronic regions of 11 of the candidate genes (excluding TAF1L, SMU1 and B4GALT1) were screened by direct sequencing of PCR products amplified from genomic template. After screening MOBKL2B, LINGO2, ACO1, and DDX58, 11 known polymorphisms (MOBKL2B: rs34959338, rs12379154; LINGO2: rs2383768, rs13296489, rs10968460; ACO1: rs34319839, rs3780473, rs35370505, rs12985; DDX58: rs3739674, rs10813831) and one novel nucleotide substitution was detected (CGT to CAT) Arg71His in DDX58. These were used to create an informative SNP haplotype to further fine map the centromeric recombination breakpoint, moving it to between D9S1118 and D9S304. This left 4 known genes (IFNK, LINGO2, MOBKL2B, C9orf11 (ACR formation associated factor)), and a hypothetical protein C9orf72. The coding and non-coding exonic sequence, and flanking intronic regions, of each of these 5 candidate genes were screened by direct sequencing of PCR products amplified from genomic template. In addition to mutation screening the coding and non-coding exons, each of the 5 genes/transcripts was analysed using two additional methods. We determined whether there were differences between normal and affected individuals in alternative or aberrant splicing by RT-PCR and agarose gel electrophoresis of lymphocyte and brain transcripts. We also determined whether there were possible differences in gene copy number in genomic DNA template by quantitative PCR using SYBR green chemistry. No coding mutations were detected in the candidate genes. No altered splicing or small-scale deletions were detected by RT-PCR of the transcripts of candidate genes. However, preliminary data suggests that there was an alteration in gene copy number of the LINGO2 and c9orf72 genes as defined by quantitative PCR, but only in individual III:8. These results indicate that III:8 is a phenocopy (i.e. the phenotype arises by means other than the inheritance of a familial gene mutation) and that the centromeric recombination breakpoint defined by individual III:8 (between D9S1118 and D9S304) is incorrect. A possible explanation of the phenocopy status of subject III:8 may be the altered gene copy number of the LINGO2 and c9orf72 genes

Re-analysis of the data was then undertaken excluding III:8, under an autosomal dominant model with 5 liability classes using the program LINKAGE and allele frequencies derived from a cohort of normal Australian individuals. Only a single region achieved a significant two-point LOD score of 3.54 for the marker D9S1817. A revised minimal disease region therefore comprises the markers D9S161 to D9S175 and spans 30 cM on chromosomal region 9p21-9q21, which overlaps all the previous reported FTD/MND linkage regions for chromosome 9.

### Example 2

### Identification of Markers in the Opioid Receptor Sigma 1 (OPRS1) Gene as Markers of Neurodegenerative Disease

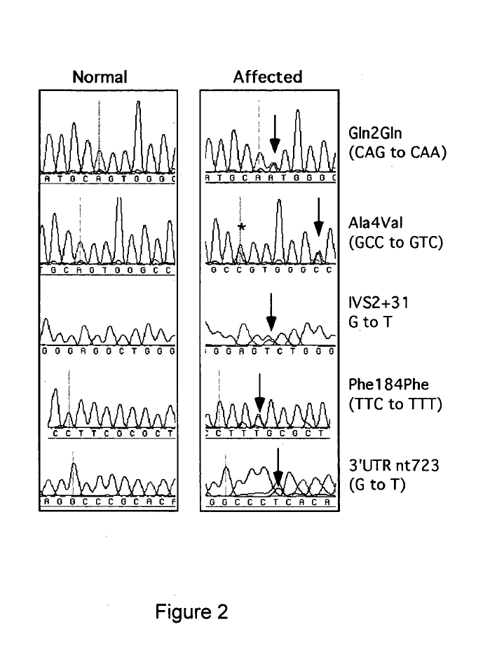

Thirty (30) genes within the revised candidate region identified in Example 1 were analyzed to determine whether or not those genes included polymorphisms or mutations associated with dementia. Those genes included UBE2R2, DNAJA1, PAX5, CNTNAP3, GDA, DNAI1, CNTFR, DCNT3, ILIIRA, GALT, CCL19, CCL21, CCL27, ARID3C, TLN1, MOBKL2B, HINT2, AQP3, UBAP1, ALDH1B1, PLAA, IFNK, P23, UNIQ470, UBAP2, TOPORS, NDUFB6, APTX, BAG1 and OPRS1. Polymorphisms were detected in several candidate genes. However, the opioid receptor sigma 1 (OPRS1) gene had a non-polymorphic nucleotide change that co-segregated with the disease phenotype in Family 14 (FIG. 2).

A G to T nucleotide change in the 3′ untranslated region of OPRS1 (nucleotide 723) was detected in the Family 14 pedigree (FIG. 2). The OPRS1 G723T change segregates with the disease haplotype in EOAD14. The G723T sequence change was not detected in a cohort of 209 elderly normal controls (from the Sydney Older Person Study SOPS cohort) indicating that it is a mutation associated with or causative of dementia.

In silico analysis of the OPRS1 3′UTR indicated that the G723T substitution is located within a conserved region of the OPRS1 transcript and is predicted to disrupt a putative stem loop structure in the transcript.

Because many nucleotide substitutions in 3′-untranslated regions have been reported to alter the stability of the cognate transcript (Cheadle et al. Ann NY Acad Sci 1058: 196-204, 2005), the relative levels of OPRS1 transcripts in lymphocytes was measured using real time RT-PCR (SyberGreen Chemistry) and normalized for cDNA levels using the house keeping GAPDH gene. OPRS1 expression levels were reduced approximately 2-fold in affected individuals from EOAD14 (n=5) and EOAD12 (n=1) compared with control individuals (n=3). This analysis indicates that a mutation in OPRS1 3′-untranslated region is associated with decreased transcript levels.

Following from this analysis a nucleic acid from subjects suffering from neurodegenerative disease were screened to identify mutations and/or polymorphisms within the OPRS1 gene that segregate with neurodegenerative disease. These subjects were from a cohort of 106 Australian early-onset presenile dementia patients, 123 subjects affected with a neurodegenerative disease from the Sydney Older Person Study (SOPS) cohort, and two cohorts from Poland comprising 160 familial cases of dementia that were negative for mutations in the APP gene, PSEN1 gene, PSEN2 gene or MAPT gene. As shown in FIG. 2 and in Table 1, four (4) additional mutations were detected in the presenile dementia cohort (a synonymous nucleotide substitution at codon position 2 (CAG to CAA; corresponding to position 2080 of SEQ ID NO: 13 or position 80 of SEQ ID NO: 5), a missense mutation resulting in a change at amino acid 4 of the OPRS1 protein from alanine to valine (this mutation occurs is a C to T mutation occurring at a position corresponding to position 2092 of SEQ ID NO: 13 or position 85 of SEQ ID NO: 5), a G to T change at position 31 (IVS2+31) (at a position corresponding to position 25783 of SEQ ID NO: 13), a synonymous nucleotide substitution at position 184 (TTC to TTT; this mutation occurs at a position corresponding to 4020 of SEQ ID NO: 13 or position 626 of SEQ ID NO: 5) in addition to the 3′ untranslated region mutation at nucleotide position 723 (G to T at a position corresponding to position 4191 of SEQ ID NO: 13 or position 1005 of SEQ ID NO: 7) found in family 14. An intronic mutation comprising a C to A change at position 24 (IVS+24; corresponding to nucleotide position 2576 of SEQ ID NO: 13) was detected an individual suffering from late-onset dementia in the SOPs cohort. Four additional nucleotide changes were identified in the Polish cohorts, in particular, a nucleotide substitution (C to G) in the 5′ UTR at position −45 (corresponding to nucleotide position 30 of SEQ ID NO: 5 or nucleotide position 2030 of SEQ ID NO: 13), an intronic mutation (T to A) in intron 3 at nucleotide position +17 (IVS3+17 T to A) (this mutation occurs at a position corresponding to nucleotide position 2792 of SEQ ID NO: 13), a synonymous nucleotide substitution at codon position 157 (GGT to GGC) (this mutation occurs at a position corresponding to nucleotide position 545 of SEQ ID NO: 5 or nucleotide position 3939 of SEQ ID NO: 13), and another 3′ UTR mutation at position 719 (G to A; at a position corresponding to nucleotide position 4187 of SEQ ID NO: 13). A cohort of 76 motor neuron disease families was then screened and 5 nucleotide changes detected, all located near exon 1. These mutations comprise the amino acid substitution Threonine to Serine at residue 23 in transmembrane domain 1 (corresponding to nucleotide position 141 of SEQ ID NO: 5 or nucleotide position 2141 of SEQ ID NO: 13), a cluster of nucleotide substitutions at intron 1. These include IVS1+29 C to A (this mutation occurs at a position corresponding to nucleotide position 2254 of SEQ ID NO: 13), IVS1+30 G to A (this mutation occurs at a position corresponding to nucleotide position 2255 of SEQ ID NO: 13), IVS1+32 C to A (this mutation occurs at a position corresponding to nucleotide position 2257 of SEQ ID NO: 13). And another 5′UTR change (−6 C to A; corresponding to nucleotide position 2070 of SEQ ID NO: 13).

TABLE 1
Mutations detected in the OPRS1 gene

| | Gene | Base | Likely |
|---|---|---|---|
| Cohort | location | change | effect |
| Polish cohort | Exon 1 | −45 C to G | (in 5′UTR) |
| MND Cohort | Exon 1 | −6 C to A | (in 5′UTR) |
| Australian Early- | Exon 1 | CAG to CAA | Gln2Gln: |
| onset dementia | | | mis-splicing |
| Australian Early- | Exon 1 | GCC to GTC | Missense: |
| onset dementia | | | Ala4Val |
| MND Cohort | Exon 1 | | Thr23Ser |
| MND Cohort | Exon 1 | IVS1 + 29 C | Mis-splicing |
| | | to A | |
| MND Cohort | Exon 1 | IVS1 + 30 G | Mis-splicing |
| | | to A | |
| MND Cohort | Exon 1 | IVS1 + 32 C | Mis-splicing |
| | | to A | |
| SOPS | Intron 2 | IVS + 24 C | Mis-splicing |
| | | to A | |
| Australian Early- | Intron 2 | IVS + 31 | Mis-splicing |
| onset dementia | | G to T | |
| Polish cohort | Intron 3 | IVS3 + 17 T | Mis-splicing |
| | | to A | |
| Australian Early- | Exon 4 | TTC to TTT | Phe184Phe: |
| onset dementia | | | mis-splicing |
| Polish cohort | Exon 4 | GGT to GGC | Glyl57Gly: |
| | | | mis-splicing |
| Polish cohort | Exon 4 | 719 G to A | (in 3′UTR) |
| Family 14 | Exon 4 | 723 G to T | (in 3′UTR) |
| (Australian | | | |
| FTLD/MND pedigree) | | | |

### Example 3

### An OPRS1 Mutation Affects OPRS1 mRNA Levels

### 3.1 Methods and Materials

A 1223 bp promoter fragment was PCR amplified from the OPRS1 gene using the oligonucleotides CTGGGGAGTAGGACCATTGTTTC (SEQ ID NO: 9) and CGTCTTCCAGCGCGAAGAGATA (SEQ ID NO: 10) and subcloned into a pGL3 vector containing the luciferase reporter gene. Consequently, a 1104 bp genomic fragment was amplified corresponding to the entire 3′-untranslated region of the OPRS1 gene using the oligonucleotides ACTGTCTTCAGCACCCAGGACT (SEQ ID NO: 11) and CTCTTGCTGTGTGATTCATGGT (SEQ ID NO: 12). Genomic DNA from subjects suffering from dementia and comprising the G723T mutant allele or from normal subjects was used as a template. Wild type and mutant alleles (G723T) were subcloned into a modified pGL3 vector containing the wildtype OPRS1 promoter. The presence of the G719A mutation was introduced into the luciferase reporter construct with the wildtype OPRS1 promoter and wildtype 3′UTR by site-directed mutagenesis.

Each recombinant vector was transfected into human neuroblastoma SK-N-MC or SK-N-SH cells using Lipofectamine 2000 reagent according to manufacture's instructions (Invitrogen). The cells were lysed after 48 hours and the levels of luciferase activity using the Readi-Glo reagent according to manufacturer's instructions (Promega).

### 3.2 Results

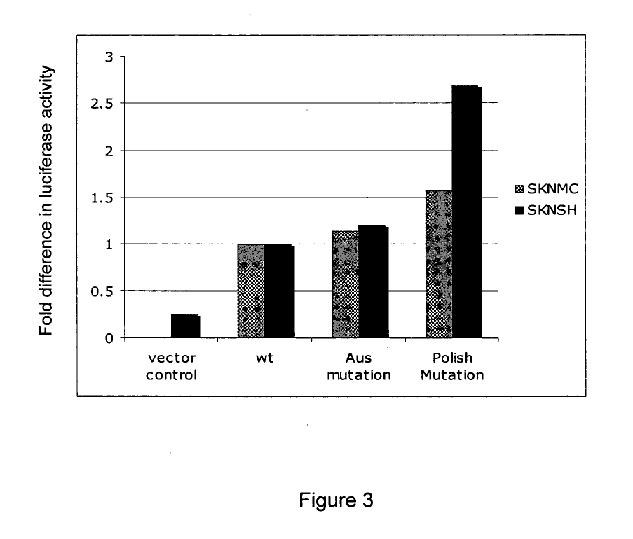

As shown in FIG. 3, both mutations increased luciferase expression in SKNMC cells and in SKNSH cells. Comparative results are shown in Table 2.

TABLE 2
Luciferase expression levels.

| | SKNMC cells | SKNSH cells | T test p value |
|---|---|---|---|
| Vector control | 0.012463 | 0.24657534 | |
| Wild type | 1 | 1 | |
| G723T (Aus. | 1.14 | 1.21 | 0.01 |
| mutation) | | | |
| G719A (Polish | 1.58 | 2.69 | 0.046 |
| mutation) | | | |

### Example 4

### A mutation in Intron 2 of OPRS1 Modulates Splicing

A 658 bp PCR product comprising exon 2 and 3 of the OPRS1 gene was amplified from genomic DNA using the primers OPRS1ExonTrapF (5′-GGAGCCTAGGGTTCCGAAG-3′; SEQ ID NO: 20) and OPRS1ExonTrapR (5′-CAACCAATCACCTGTGGCTTATG-3′; SEQ ID NO: 21). Genomic DNA from subjects suffering from dementia and comprising the IVS+31 or UVS+24 mutant alleles or from normal subjects was used as a template. Wild type and mutant alleles (IVS+31 or IVS+24) were subcloned into the exon trap vector pSPL3 (Gibco BRL, CA). Each recombinant vector was transfected into the human neuroblastoma cell line, SK-N-MC (ATCC HTB 10) or human embryonic kidney 293 cells (ATCC CRL 1573) using Lipofectamine 2000 (Invitrogen). Cells were left for 48 hours before total RNA was extracted and the exon trap products detected by RT-PCR essentially as described previously in Stanford et al Brain; 123: 880-893, 2000.

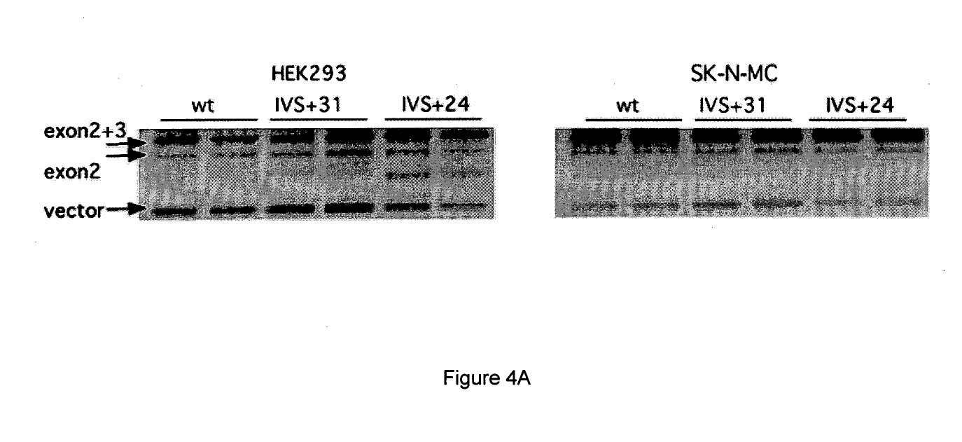

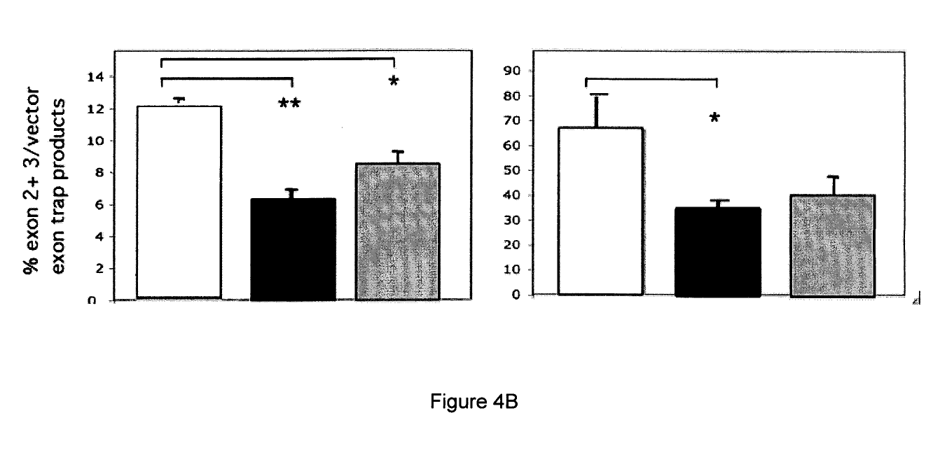

As shown in FIGS. 4A and 4B both IVS1+24 and IVS2+31 increase the level of alternative splicing of OPRS1, and significantly reduce the level of correctly spliced OPRS1 mRNA. Accordingly, these results provide additional markers for diagnosing a neurodegenerative disease or determining a predisposition to a neurodegenerative disease or predicting an increased risk of developing a neurodegenerative disease, e.g., by virtue of detecting a reduced level of wild type OPRS1 and/or by detecting an increased level of or the presence of alternatively spliced OPRS1.

### Example 5

### OPRS1 Mutations Increase Gamma-Secretase Activity

The presence of a FLAG motif at the amino-terminal end of the OPRS1 protein was introduced using the primers OPRS1-FLAGF (5′-AAAAGCTTATGGATTACAAGGATGACGACGATAAGCAGTGGGCCGTGGGC-3′; SEQ ID NO: 18) and OPRS1-FLAGR (5′-AGGATCCTGGTGGGGAGGAGGTGGGAA-3′; SEQ ID NO: 19) to generate the pCDNA-FLAG-OPRS1 (wt) plasmid. Site-directed mutagenesis was used to add either the rs1800866 polymorphism or the Ala4Val mutation into the pCDNA-FLAG-OPRS1 (wt) plasmid, to generate the pCDNA-FLAG-OPRS1 (rs1800899) and pCDNA-FLAG-OPRS1 (Ala4Val) plasmid respectively.

Gamma-secretase activity was measured using a luciferase reporter assay essentially as described in Karlstrom et al. Journal of Biological Chemistry, 277: 6763-6766 2002. Briefly, two reporter constructs (MH100 and C99-GVP plasmids) are co-transfected with the OPRS1 expression constructs into the human neuroblastoma cell line, SK-N-MC (ATCC HTB 10) or into SK-N-SH cells (ATCC HTB 11) using Lipofectamine 2000 (Invitrogen). The cells were lysed after 48 hours and the levels of luciferase activity using the Readi-Glo reagent according to manufacturer's instructions (Promega).

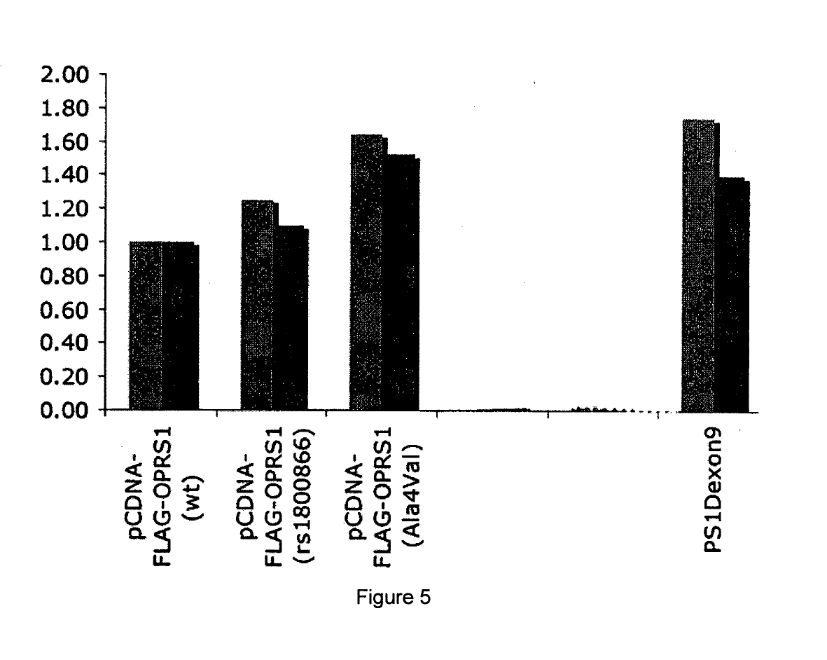

As shown in FIG. 5, the level of gamma secretase activity was significantly increased in cells expressing the Ala4Val mutation compared to cells overexpressing native OPRS1. The level of gamma secretase activity was comparable to that detected in cells expressing the presenillin 1 Δexon 9 mutation, which is known to increase gamma-secretase activity in subjects suffering from AD.

### Example 6

### Effect of OPRS1 Mutations on Tau Phosphorylation

### 6.1 Construction of Expression Constructs

Nucleic acid comprising each of the mutations identified in OPRS1 that are transcribed and expressed as identified in Example 3 are amplified by PCR using lymphocyte cDNA. Each PCR product is subcloned into the mammalian expression vector pCDNA3.1 (Invitrogen). Additionally, a vector is produced comprising an OPRS1 cDNA placed under control of an OPRS1 promoter and mutant OPRS1 3′ untranslated region.

As a control, a vector is produced comprising an OPRS1 cDNA placed under control of an OPRS1 promoter and wild-type OPRS1 3′ untranslated region.

COS-7 cells are then transfected with the gene constructs.

### 6.2 Detection of Tau Species

Transfected cells are lysed in 1× Lysis buffer (50 mM Tris.HCl (pH 7.4), 150 mM NaCl, 1 mM PMSF, 1× complete cocktail protease inhibitor (Boehringer Mannheim) and 0.05% Triton X-100. Approximately 2-25 μg of total protein is used to assay for total Tau or Tau phosphorylated at serine residue 396 using the Human Tau or Human Tau [pS396] ELISA kit respectively (Biosource International, CA, USA).

### 6.3 Results

The ability of each form of OPRS1 to phosphorylate Tau at a serine residue 396 (Tau [pSer396]) is examined. COS-7 cells are transfected with each cDNA and endogenous Tau phosphorylation is measured by ELISA. For example, the level of Tau phosphorylation is determined in cells comprising each of the mutations descried herein relative to control cells. Mutations associated with increased Tau phosphorylation, a characteristic of Alzheimer's disease are then identified.

### Example 7

### Effect of OPRS1 Agonists on Mutant Forms of OPRS1

### 7.1 Production of Cells Expressing Specific OPRS1 Isoforms

COS-7 cells are plated onto 12 well plates at concentration of 1×10⁵ cells/well and allowed to recover for 24 hours. Each well is transfected with each of the vectors described in Example 5 using Lipofectamine 2000. After 48 hours, growth media are removed and cells exposed to pregnenolone sulphate or SA4503 (1-(3,4-dimethoxyphenethyl)-4-(3-phenylpropyl)piperazine dihydro-chloride) (Senda et al., Eur. J. Pharmacol., 315: 1-10, 1996) or 2-(4-morpholinethyl)1-phenylcyclohexanecarboxylate (Marrazzo et al., NeuroReport 16: 1223-1226, 2005) serially diluted in growth medium. Media are removed, cells lysed in situ, and the level of endogenous Tau [pS396] phosphorylation measured as described above.

### 8.2 Results

To gain biologically relevant insights into the actions of OPRS1 agonists in dementia, the ability of various OPRS1 agonists to inhibit phosphorylation of endogenous Tau protein is examined in living COS-7 cells that express mutant forms of OPRS1.

### Example 8

### OPRS1 Gene Expression in Subjects Suffering from Neurodegeneration and Carrying the 3′ UTR G723T Mutation

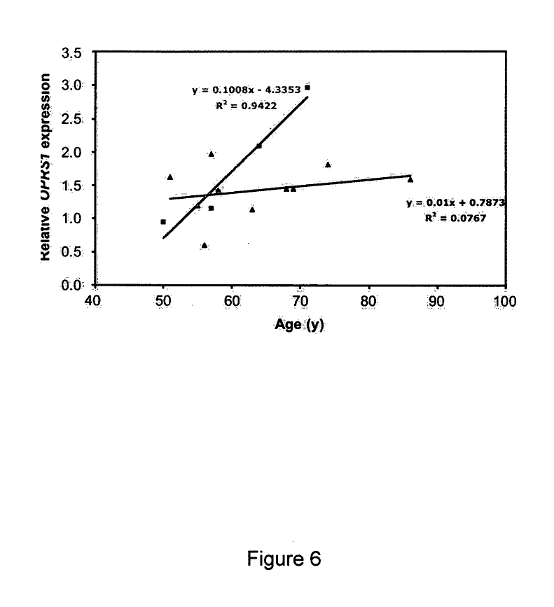

Total RNA was extracted from immortalized lymphocytes and reversed transcribed using a poly-dT primer. OPRS1 transcript levels were determined by SYBR green chemistry quantitative PCR using primer OPRS1-RTF (5′-ACCATCATCTCTGGCACCTT-3′; SEQ ID NO: 22) and OPRS1-RTR (5′-CTCCACCATCCATGTGTTTG-3′; SEQ ID NO: 23). Transcript levels between samples were normalized using primers that amplify the house-keeping gene, succinate dehydrogenase complex, subunit A (SDHA) essentially as described in Vandesompele et al. Genome Biology 3, 2002. As shown in FIG. 6, there is a strong correlation between normalized OPRS1 transcript levels and the age of the individual when their lymphocytes were immortalized, in affected individuals (r2=0.9422), but not in control lymphocytes (r2=0.0767). Linear regression analyses indicated that ‘age x disease status’ interaction was a significant predictor of OPRS1 cDNA levels (β=4.995, t=3.713, p=0.004). There is an overall increase in OPRS1 transcript levels (1.13 fold) in affected individuals compared with unaffected controls.

### Example 9

### Increase in OPRS1 Transcript Levels is Correlated with Increased the Level of the TAR DNA Binding Protein-43 (TDP-43) in the Cytoplasm of Lymphocyte Cell Lines from 3′UTRG723T Mutation Carriers

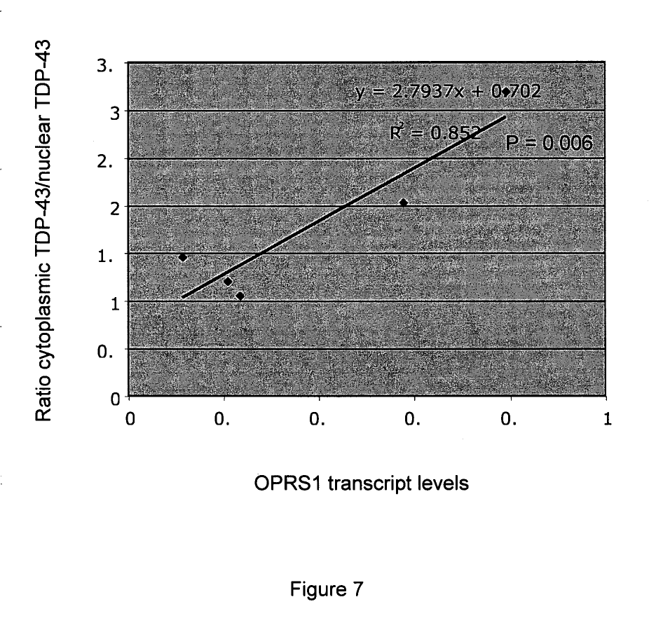

Cytoplasmic and nuclear subcellular fractions were isolated sequentially from lymphocyte cell lines using the Proteoextract Subcellular Proteome Extraction Kit (Calbiochem, La Jolla, Calif., USA) according to manufacturer's instructions. Approximately 10 μg of protein lysates were heated to 95° C. for 10 minutes prior to electrophoresis on a 7.5% SDS-PAGE gel and transferred to a nitrocellulose membrane (Trans-blot transfer medium, Biorad, CA). A rabbit polyclonal antibody (Proteintech Group Inc, Chicago, Ill., USA) was used to detect the TDP-43 protein. Densities of chemiluminescence bands were quantified using the Biorad Chemidoc system. Results show a strong correlation between OPRS1 transcript levels and the relative amount of TDP-43 protein in the cytoplasm as expressed as a ratio of TDP-43 in cytoplasmic versus nuclear fraction. Densities of chemiluminescence bands were quantified using the Biorad Chemidoc system. As shown in FIG. 7, there is a strong correlation (r2=0.852, p=0.006) between OPRS1 transcript levels and the relative amount of TDP-43 protein in the cytoplasm as expressed as a ratio of TDP-43 in cytoplasmic versus nuclear fraction.

### Example 10

### Overexpression of OPRS1 cDNAs Increases the Level of the TAR DNA Binding Protein (TDP-43) in the Cytoplasm of Two Transfected Neuronal Cell Lines

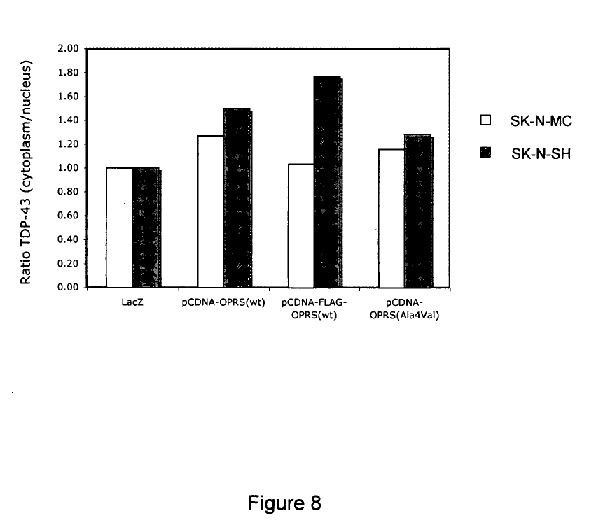

A full-length wildtype OPRS1 cDNA was constructed by RT-PCR of lymphocyte RNA using the primers OPRS1-RTF (5′-AAAAGCTTATGCAGTGGGCCGTGGGC-3′; SEQ ID NO: 24) and OPRS1-RTR (5′-AGGATCCTGGTGGGGAGGAGGTGGGAA-3′; SEQ ID NO: 25), and subcloned into the expression vector pCDNA3.1 (Invitrogen) to generate the pCDNA-OPRS1 (wt) plasmid. The presence of the Ala4Val mutation was introduced into the OPRS1 expression construct by site-directed mutagenesis to generate the pCDNA-OPRS1 (Ala4Val) plasmid. The presence of a FLAG motif at the amino-terminal end of the OPRS1 protein was introduced using the primers OPRS1-FLAGF (5′-AAAAGCTTATGGATTACAAGGATGACGACGATAAGCAGTGGGCCGTGGGC-3′; SEQ ID NO: 26) and OPRS1-FLAGR (5′-AGGATCCTGGTGGGGAGGAGGTGGGAA-3′; SEQ ID NO: 27) to generate the pCDNA-FLAG-OPRS1 (wt) plasmid. Each recombinant vector was transfected into the human neuroblastoma cell line, SK-N-MC (ATCC HTB 10) and SK-N-SH cells (ATCC HTB 11) using Lipofectamine 2000 (Invitrogen). Cells were left for 48 hours prior to western blot analyses of TDP-43 protein levels. Cytoplasmic and nuclear subcellular fractions were isolated sequentially from transfected cells using the Proteoextract Subcellular Proteome Extraction Kit (Calbiochem, La Jolla, Calif., USA) according to manufacturers. Approximately 10 μg of protein lysates were heated to 95° C. for 10 minutes prior to electrophoresis on a 7.5% SDS-PAGE gel and transferred to a nitrocellulose membrane (Trans-blot transfer medium, Biorad, CA). A rabbit polyclonal antibody (Proteintech Group Inc, Chicago, Ill., USA) was used to detect the TDP-43 protein. Densities of chemiluminescence bands were quantified using the Biorad Chemidoc system. As shown in FIG. 8, the over-expression of the wildtype OPRS1 cDNA in transfected cells significantly increase (1.3 to 1.5 fold, p=0.019, Student's t test) the level of TDP-43 in the cytoplasm compared to cells transfected with the control LacZ vector.

### Example 11

### Preparation of a Monoclonal Antibody that Recognizes an OPRS Ala4Val Mutant Polypeptide

A monoclonal antibody that specifically binds to an epitope of OPRS1 comprising the Ala4Val mutation is produced using methods known in the art. Briefly, a peptide antigen that corresponds to the region of OPRS1 comprising the Ala4Val mutation is synthesized essentially using the methods described in Bodanszky, M. (1984) Principles of Peptide Synthesis, Springer-Verlag, Heidelberg and Bodanszky, M. & Bodanszky, A. (1984) The Practice of Peptide Synthesis, Springer-Verlag, Heidelberg.

Peptides are purified using HPLC and purity assessed by amino acid analysis.

Female BalB/c mice are immunized with a purified form of the peptide. Initially mice are sensitized by intraperitoneal injection of Hunter's Titermax adjuvant (CytRx Corp., Norcross, Ga.). Three boosts of the peptide are administered at 2, 5.5 and 6.5 months post initial sensitization. The first of these boosts is a subcutaneous injection while the remaining are administered by intraperitoneal injection. The final boost is administered 3 days prior to fusion.

The splenocytes of one of the immunized BALB/c mice is fused to X63-Ag8.653 mouse myeloma cells using PEG 1500. Following exposure to the PEG 1500 cells are incubated at 37° C. for 1 hour in heat inactivated fetal bovine serum. Fused cells are then transferred to RPMI 1640 medium and incubated overnight at 37° C. with 10% CO₂. The following day cells are plated using RPMI 1640 media that has been supplemented with macrophage culture supernatants.

Two weeks after fusion, hybridoma cells are screened for antibody production by solid phase ELISA assay. Standard microtitre plates are coated with recombinant OPRS1 Ala4Val in a carbonate based buffer. Plates are then blocked with BSA, washed and then the test samples (i.e. supernatant from the fused cells) is added, in addition to control samples, (i.e. supernatant from an unfused cell). Antigen-antibody binding is detected by incubating the plates with goat-anti-mouse HRP conjugate (Jackson ImmunoResearch Laboratories) and ABTS peroxidase substrate system (Vector Laboratories, Burlingame, Calif. 94010, USA). Absorbance is read on an automatic plate reader at a wavelength of 405 nm.

Any colonies that are identified as positive by these screens continue to be grown and screened for several further weeks. Stable colonies are then isolated and stored at 80° C.

Positive stable hybridomas are then cloned by growing in culture for a short period of time and diluting the cells to a final concentration of 0.1 cells/well of a 96 well tissue culture plate. These clones are then screened using the previously described assay. This procedure is then repeated in order to ensure the purity of the clone.

Four different dilutions, 5 cells/well, 2 cells/well, 1 cell/well, 0.5 cells/well of the primary clone are prepared in 96-wells microtiter plates to start the secondary cloning. Cells are diluted in IMDM tissue culture media containing the following additives: 20% fetal bovine serum (FBS), 2 mM L-glutamine, 100 units/ml of penicillin, 100 μg/ml of streptomycin, 1% GMS-S, 0.075% NaHCO₃. To determine clones that secrete anti-human OPRS1 antibody, supernatants from individual wells of the 0.2 cells/well microtiter plate are withdrawn after two weeks of growth and tested for the presence of antibody by ELISA assay as described above.

All positive clones are then adapted and expanded in RPMI media containing the following additives: 10% FBS, 2 mM L-glutamine, 100 units/ml of penicillin, 100 μg/ml of streptomycin, 1% GMS-S, 0.075% NaHCO₃, and 0.013 mg/ml of oxalaacetic acid. A specific antibody is purified by Protein A affinity chromatography from the supernatant of cell culture.

The titer of the antibodies produced using this method are determined using the Easy Titer kit available from Pierce (Rockford, Ill., USA). This kit utilizes beads that specifically bind mouse antibodies, and following binding of such an antibody these beads aggregate and no longer absorb light to the same degree as unassociated beads. Accordingly, the amount of an antibody in the supernatant of a hybridoma is assessed by comparing the OD measurement obtained from this sample to the amount detected in a standard, such as for example mouse IgG.

The specificity of the monoclonal antibody is then determined using a Western blot.

### Example 12

### Determining the Level of OPRS1 Ala4Val in a Biological Sample

A monoclonal antibody that binds to the OPRS1 Ala4Val mutant as described in Example 11 is used in the production of a two-site ELISA to determine the level of mutant OPRS1 in a biological sample.

A polyclonal antibody that binds to OPRS1 is adsorbed to a microtitre plate at 20° C. for 16 hours. Plates are then washed and blocked for 1 hour. Recombinant OPRS1 Ala4Val is serially diluted, added to wells of the microtitre plate and incubated for 1 hour.

Alternatively, serum from patients suffering from FTLD is diluted in PBS and added to wells comprising the antibody.

The monoclonal antibody described in Example 11 is conjugated to horseradish peroxidase (HERP) using a HRP conjugation kit (Alpha Diagnostics International, Inc., San Antonio, Tex., USA).

Following washing of the microtitre plates, the HRP conjugated monoclonal antibody is added to each well of the plate and incubated. Plates are then washed and ABTS (Sigma Aldrich, Sydney, Australia) is added to each well. Reactions are stopped after approximately 20 minutes and absorbance values measured at 415 nm.

The amount of absorbance detected in negative control wells (no OPRS1 Ala4Val or patient serum added) is subtracted from the absorbance of each other well to determine the amount of antibody bound to OPRS1.

1.-3. (canceled)

4. A method for diagnosing a neurodegenerative disease in a subject or determining the predisposition of a subject to developing a neurodegenerative disease or determining an increased risk of a subject developing a neurodegenerative disease, the method comprising detecting in a sample from the subject a marker within an opioid receptor sigma 1 (OPRS1) gene or an expression product thereof that is associated with or linked or causative of a neurodegenerative disease, wherein detection of said marker is indicative of a neurodegenerative disease or a predisposition to a neurodegenerative disease or an increased risk of developing a neurodegenerative disease.

5. The method according to claim 4, wherein the neurodegenerative disease is a dementia or a motor neuron disease.

6. The method according to claim 5 wherein the dementia is an Alzheimer's disease.

7. The method according to claim 5 wherein the dementia is frontotemporal lobar dementia.

8. The method according to claim 5 wherein the motor neuron disease is amyotrophic lateral sclerosis (ALS).

9. The method according to claim 4 wherein the marker comprises a mutation in an OPRS-1 genomic gene and/or an expression product thereof.

10. The method according to claim 4, wherein the marker is associated with or causes alternative splicing of an OPRS1 mRNA.

11. (canceled)

12. The method according to claim 4, wherein the marker is associated with or causes increased expression of an OPRS1 transcript.

13.-14. (canceled)

15. The method according to claim 4, wherein the marker is within an OPRS1 polypeptide.

16. (canceled)

17. The method according to claim 4, wherein the method comprises hybridizing a nucleic acid probe comprising the sequence of the marker to nucleic acid in a sample from a subject under moderate to high stringency hybridization conditions thereby forming a complex between the probe and the sample nucleic acid and detecting the complex using a detection means, wherein complex formation and detection indicates that the subject suffers from a neurodegenerative disease or a has a predisposition to a neurodegenerative disease or has an increased risk of developing a neurodegenerative disease.

18. The method according to claim 4, wherein a marker in an OPRS1 expression product is in an OPRS1 polypeptide and said method comprises contacting a biological sample derived from a subject with an antibody or ligand that binds specifically to said OPRS1 thereby forming an antibody/ligand complex or a ligand/ligand complex and then detecting the complex using a detection means, wherein complex formation and detection indicates that the subject being tested suffers from a neurodegenerative disease or a has a predisposition to a neurodegenerative disease or has an increased risk of developing a neurodegenerative disease.

19. The method according to claim 4, wherein the marker is detected by determining an enhanced or reduced level of an OPRS1 transcript in a sample from the subject, wherein said enhanced or reduced level of the OPRS1 transcript is indicative of a neurodegenerative disease or a predisposition to a neurodegenerative disease or an increased risk of developing a neurodegenerative disease.

20. The method according to claim 19 wherein an enhanced or reduced level of an OPRS1 transcript is detected by performing a process comprising:
(i) determining the level of the OPRS1 transcript in a sample from the subject;
(ii) determining the level of the OPRS1 transcript in a suitable control sample,
wherein an enhanced or reduced level of the OPRS1 transcript at (i) compared to (ii) is indicative of a neurodegenerative disease or a predisposition to a neurodegenerative disease or an increased risk of developing a neurodegenerative disease.

21. The method according to claim 20 wherein the level of the OPRS1 transcript is determined by performing a process comprising hybridizing a nucleic acid probe that selectively hybridizes to the OPRS1 transcript to nucleic acid in a sample from the subject under moderate to high stringency hybridization conditions thereby forming a complex between the probe and the OPRS1 transcript and detecting the level of hybridization using a detection means, wherein a level of nucleic acid complex formation and detection is indicative of the level of the OPRS1 transcript in the sample.

22. The method according to claim 4, wherein the marker is detected by determining an enhanced or reduced level of an OPRS1 polypeptide in a sample from the subject, wherein said enhanced or reduced level of the OPRS1 polypeptide is indicative of a neurodegenerative disease or a predisposition to a neurodegenerative disease or an increased risk of developing a neurodegenerative disease.

23. (canceled)

24. The method according to claim 22 wherein detecting an enhanced or reduced level of the OPRS1 polypeptide comprises performing a process comprising:
(i) determining the level of the OPRS1 polypeptide in a sample from the subject;
(ii) determining the level of the OPRS1 polypeptide in a suitable control sample,
wherein an enhanced or reduced level of the OPRS1 polypeptide at (i) compared to (ii) is indicative of a neurodegenerative disease or a predisposition to a neurodegenerative disease or an increased risk of developing a neurodegenerative disease.

25. The method according to claim 24 wherein the level of the OPRS1 polypeptide is detected by performing a process comprising contacting a biological sample derived from the subject with an antibody or ligand that binds selectively to the OPRS1 polypeptide thereby forming an antibody/ligand complex or ligand/ligand complex and then detecting the complex using a detection means, wherein a level of complex formation and detection is indicative of the level of the OPRS1 polypeptide in the subject.

26. (canceled)

27. A method of treatment or prophylaxis of a neurodegenerative disease, said method comprising:
(i) performing the method according to claim 4 to thereby diagnose a neurodegenerative disease in a subject or determine a predisposition or increased risk of a subject to developing a neurodegenerative disease; and
(ii) administering or recommending a therapeutic or prophylactic compound for the treatment of the neurodegenerative disease.

28. (canceled)

29. A method for predicting the response of a subject to treatment with a composition for the treatment or prophylaxis of a neurodegenerative disease, said method comprising detecting a marker within an OPRS-1 gene or an expression product thereof that is associated with response of a subject to treatment with a composition for the treatment or prophylaxis of a neurodegenerative disease, wherein detection of said marker is indicative of the response of the subject to treatment with said composition.

30. A method for identifying a marker in an OPRS-1 gene or expression product that is associated with a neurodegenerative disease, said method comprising:
(i) identifying a polymorphism or allele or mutation within an OPRS-1 gene or expression product thereof;
(ii) analyzing a panel of subjects to determine those that suffer from a neurodegenerative disease, wherein not all members of the panel comprise the polymorphism or allele or mutation; and
(iii) determining the variation in the development of the neurodegenerative disease wherein said variation indicates that the polymorphism or allele or mutation is associated with the neurodegenerative disease or a subject's predisposition to the neurodegenerative disease.

31. An isolated nucleic acid comprising a sequence selected from the group consisting of:
(i) a sequence set forth in SEQ ID NO: 7, wherein the sequence comprises a thymine at a position corresponding to nucleotide position 1005 of SEQ ID NO: 7;
(ii) a sequence set forth in SEQ ID NO: 5, wherein the sequence comprises an adenosine at a position corresponding to nucleotide position 80 of SEQ ID NO: 5 and/or a thymine at a position corresponding to position 85 of SEQ ID NO: 5 and/or an adenosine at a position corresponding to nucleotide position 626 of SEQ ID NO: 5;
(iii) a sequence set forth in SEQ ID NO: 8, wherein the sequence comprises a thymine at a position corresponding to nucleotide position 699 of SEQ ID NO: 8;
(iv) a sequence set forth in SEQ ID NO: 13, wherein the sequence comprises a an adenosine at a position corresponding to position 2080 of SEQ ID NO: 13 and/or a thymine at a position corresponding to position 2092 of SEQ ID NO: 13 and/or a thymine at a position corresponding to position 2583 of SEQ ID NO: 13 and/or a thymine at a position corresponding to position 4020 of SEQ ID NO: 13 and/or a thymine at a position corresponding to position 4191 of SEQ ID NO: 13 and/or an adenosine at a position corresponding to position 4187 of SEQ ID NO: 13;
(v) a combination of any of (i) to (iv); and
(vi) a sequence complementary to any one of (i) to (v).

32.-33. (canceled)

34. An isolated OPRS1 protein comprising a sequence set forth in SEQ ID NO: 6 wherein the sequence comprises a valine at a position corresponding to position 4 of SEQ ID NO: 6 and/or a serine at a position corresponding to position 23 of SEQ ID NO: 6.

35. An isolated antibody or antigen binding fragment thereof capable of preferentially or specifically binding to a polypeptide comprising a sequence set forth in SEQ ID NO: 6 wherein the sequence comprises a valine at a position corresponding to position 4 of SEQ ID NO: 6 or a sequence set forth in SEQ ID NO: 6 wherein the sequence comprises a serine at a position corresponding to position 23 of SEQ ID NO: 6.

36. The method of claim 4 further comprising isolating, obtaining or providing the sample.


SEQUENCE LISTING (27 sequences)

SEQ ID NO: 1 - DNA, 138 bp, Homo sapiens
agctttctatgctgcataacaaattaccacaaacttaccaacttgaaacacacacacacacacacacacacacacggacacacacacacatatttattatctgtttccggggatcaggagtctaggcatgtcttagct

SEQ ID NO: 2 - DNA, 331 bp, Homo sapiens
  misc_feature (196)..(196): n is a, c, g, or t
ataaataaataaacaatatatgtaatgtgctaaataccagagttgaaagatgttctgagtactatatgaacatgaaagatagcagtaagttcttcctggagagagacaaagggacagcatcaaagacacacacacacacacacacacacacacacacacacacagactgaatggctgataagagtttgtgaggtanaaatgttgggagagggcattctaggtaaggggnacagcataaaaaataagtcatgaactttatattccatactctatacgggtctctgtctacaagccatgactacatgtatgtgtttaagttgctgtgtaagct

SEQ ID NO: 3 - DNA, 203 bp, Homo sapiens
  misc_feature (18)..(18): n is a, c, g, or t
agctgggttatttaagtnaagttttatgtgaacacacatgcgcgcgcgcacacacacacacacacacacacacacacacacattgttaagcctccaatgttgctcctcagggaggcatggctttggggatggctacagtaaccctgggatgataataggattggtaggactctttttctttccttccctgaccacacccagct

SEQ ID NO: 4 - DNA, 301 bp, Homo sapiens
  misc_feature (72)..(72): n is a, c, g, or t
agctgtagtgagccctgattgcaccactgccactgcactccagcctggtcatagagagagatcctgtctctntctctctctctctctctctcacacacacacacacacacacacacanacacacacacacacacaaaaggcttagatanccacaaggnctgtatgttaaacattaaacctacatttaaatanccaaatcatattttcttgaatccaaaattattctgaaaaacaaaacaaagaantttaaagtctcaaggctcctaacgtcacaacantgcttctgtaaaaattttcagct

SEQ ID NO: 5 - DNA, 1655 bp, Homo sapiens
  misc_feature (30)..(30): S = C or G
ctccgaggccgtgagcgcaaagcctcaggscccggctccctcctgagctgcgccgtgccaggccgcccgccgggatgcartgggycgtgggccggcggtgggcgtgggccgcgctgctcctggctgtcgcagcggtgctgwcccaggtcgtctggctctggctgggtacgcagagcttcgtcttccagcgcgaagagatagcgcagttggcgcggcagtacgctgggctggaccacgagctggccttctctcgtctgatcgtggagctgcggcggctgcacccaggccacgtgctgcccgacgaggagctgcagtgggtgttcgtgaatgcgggtggctggatgggcgccatgtgccttctgcacgcctcgctgtccgagtatgtgctgctcttcggcaccgccttgggctcccgcggccactcggggcgctactgggctgagatctcggataccatcatctctggcaccttccaccagtggagagagggcaccaccaaaagtgaggtcttctacccaggggagacggtagtacacgggcctggygaggcaacagctgtggagtgggggccaaacacatggatggtggagtacggccggggcgtcatcccatccaccctggccttygcgctggccgacactgtcttcagcacccaggacttcctcaccctcttctatactcttcgctcctatgctcggggcctccggcttgagctcaccacctacctctttggccaggacccttgaccagccaggcctgaaggaagacctgcggatggacaggagcgggcaggcccgcacatatccacttgctggagcccatgtttacagacagggacatacaccatgcagatcctgagttcctgctgtatgagcagggatatccatgcttatgtatccaaacacagagacccatgggaacaaatgagacacatatagatactgagacctgtgtgtacagtaggaccatgcactcacacccatctggagagggagcccccggtataccaagggagccagttgtgttcagacacacacatcacagcttgactcactaactgaggcctttccatagctccacagcttcccacctcctccccaccaaaccggggttctagagttaaggatgggggagggtattatactgcctcagtctgactcctcaacccagcagcaatttgaggggatgagggggaagaggagctgccttttggaggcccccttcacctgcagctatgatgcccttccccttctcccctgtcctcaccatatgccttatccccattctactcccctgctatgcaagtgcccctgtggcttgtccccaaccccctcagcaacaaagctcagctggggaacgagagtaatttgaagaatgcttgaagtcagcgtcttccattccagaaagacccccattcttcctttgggggtatgatgtggaagctggtttcagcccaggacccaccactgaggagaggatctagacaggtgggcctaattccaaggggcccktcctggcctggagaaggccttttacacacacacaacacatacacacacacacacacacacacatatcacagttttcacacagcccctgctgcattctctgtccatctgtctgtttctattaataaagatttgttgatctgttcc

SEQ ID NO: 6 - protein, 223 aa, Homo sapiens
  misc_feature (4)..(4): The ′Xaa′ at location 4 stands for Ala, or Val.
MQWXVGRRWAWAALLLAVAAVLXQVVWLWLGTQSFVFQREEIAQLARQYAGLDHELAFSRLIVELRRLHPGHVLPDEELQWVFVNAGGWMGAMCLLHASLSEYVLLFGTALGSRGHSGRYWAEISDTIISGTFHQWREGTTKSEVFYPGETVVHGPXEATAVEWGPNTWMVEYGRGVIPSTLAFALADTVFSTQDFLTLFYTLRSYARGLRLELTTYLFGQDP

SEQ ID NO: 7 - DNA, 1400 bp, Homo sapiens
ccctgtcccccaggggagacggtagtacacgggcctggtgaggcaacagctgtggagtgggggccaaacacatggatggtggagtacggccggggcgtcatcccatccaccctggccttcgcgctggccgacactgtcttcagcacccaggacttcctcaccctcttctatactcttcgctcctatgctcggggcctccggcttgagctcaccacctacctctttggccaggacccttgaccagccaggcctgaaggaagacctgcggatagacaggagcgggcaggcccgcacatatccacttgctggagcccatgtttacagacagggacatacaccatgcagatcctgagttcctgctgtatgagcagggatatccatgcttatgtatccaaacacagagacccatgggaacaaatgagacacatatagatactgagacctgtgtgtacagtaggaccatgcactcacacccatctggagagggagcccccggtataccaagggagccagttgtgttcagacacacacatcacagcttgactcactaactgaggcctttccatagctccacagcttcccacctcctccccaccaaaccggggttctagagttaaggatgggggagggtattatactgcctcagtctgactcctcaacccagcagcaatttgaggggatgagggggaagaggagctgccttttggaggcccccttcacctgcagctatgatgcccttccccttctcccctgtcctcaccatatgccttatccccattctactcccctgctatgcaagtgcccctgtggcttgtccccaaccccctcagcaacaaagctcagctggggaacgagagtaatttgaagaatgcttgaagtcagcgtcttccattccagaaagacccccattcttcctttgggggtatgatgtggaagctggtttcagcccaggacccaccactgaggagaggatctagacaggtgggcctaattccaaggggcccktcctggcctggagaaggccttttacacacacacaacacatacacacacacacacacacacacatatcacagttttcacacagcccctgctgcattctctgtccatctgtctgtttctattaataaagatttgttgatctgttccatctttttgtgtgtctgagcagccggaaaaagcctctgacccctggggacctcaggaagatctctctcttgctgtgtgattcatggtagattttttttttttttttttaagagatggggtctcctctgttgcccaggctggagtgcagtggcacagtgacatgatcatagctccctgcagccttgaactcatggtcccaaacaattttcctgccttagccttatgagtagctgggaccttatgagtagcaa

SEQ ID NO: 8 - DNA, 1224 bp, Homo sapiens
ctggggagtaggaccattgtttctagccatatccctaatggccaccacagaacttggcacatacaagtgccaacaaagtatctgaatgaatgacaactcccagctctatctccagactggagctgagcttcgagacctggacactcaactgcccactagacatgcaaaggatgttagccaaataaggggaaagtggggataggataaagggggaaagagtattccaggaagaagagatcaagccctgccaactccatttccagagttgttaagctcatttctttcttccatctccactgacacagaccttccctcttacctggcccacagtcatggtctccctgactcatcttgctcccctccactcgacagtcctgtgccactcggtggcaaaatctaatccccttcctcccttgctgataaccctcagctggtttacaaggcctcctctcacattttgtgccgcagaacagagaaggaattgtaggcccctgaagaccccaagctgtctctcctcggaggcctttgctctaggggctggtccctgtgggctggcatgccccttctgcgagctcttaaagacactgctcaggtgtcaccgcctctggaatgcccaccctggggctcccagcttgaactgcctcctctccgcttcagtcctggtaaaggcgggccttaacaatcgttgagtcccagaaggcttcacttccgggctcgtctgcttcaccagccagtgagctcgggcgagcccgtccattctgggtctcactatccccacacctatccgtatgtagagtggcgctagctaggtcagacctcgcctgaagaccctgacatctgccgctgggcgacttggccccgcccccttgcctcccttgcgcggcaccgccccactcctccgtgctccccccgaggaaatggttcaaccgaagggcggtgccaaggtgcccgggccgctccgattggtcagggcgagccgtaccacggcggtggcgggggagcgcttcgtgggcagccggcgggctccgaggccgtgagcgcaaagcctcaggccccggctccctcctgagctgcgccgtgccaggccgcccgccgggatgcagtgggccgtgggccggcggtgggcgtgggccgcgctgctcctggctgtcgcagcggtgctgacccaggtcgtctggctctggctgggtacgcagagcttcgtcttccagcgcgaagagatag

SEQ ID NO: 9 - DNA, 23 bp, Artificial Sequence
  Oligonucleotide for amplifying OPRS1 promoter region
ctggggagtaggaccattgtttc

SEQ ID NO: 10 - DNA, 22 bp, Artificial Sequence
  Oligonucleotide for amplifying OPRS1 promoter region
cgtcttccagcgcgaagagata

SEQ ID NO: 11 - DNA, 22 bp, Artificial Sequence
  Oligonucleotide for amplifying OPRS1 3′- untranslated region
actgtcttcagcacccaggact

SEQ ID NO: 12 - DNA, 22 bp, Artificial Sequence
  Oligonucleotide for amplifying OPRS1 3′- untranslated region
ctcttgctgtgtgattcatggt

SEQ ID NO: 13 - DNA, 7049 bp, Homo sapiens
  misc_feature (2030)..(2030): S = C or G
ctgcttcagcctcccgagtagctgggactacaggcacctgccaccacgcccggctaattttttgtattttagtagagacgaggtttcaccatgttgcccagggtggtctcgaacttctgagctcaggaaatctgcctgcccaccttggcctcccaaagtgctgggactacaggtgtgtgccactgtgcctggccctttttttttttttttttttttttttttttgaggcagagtctccctctgttggctcaactgcaacctccacttcccaggctcaagcgatcctcccacttcggcctcctgagtagctgggactacaggtgtgcgcccccactcctgactaatttttgtactttttgcagagatggggtttcaccatgttgccaaggctggtctcgaatacctggactcaagtgttccgtctgcctcagcctcccaaagtgttgggattacagttatgagccacctcgcccggcctcagtttctttctttaaggtcaaaaatgtatgaggctacttttttccagttcctttatgttttatatatataaccatcacataaaattctcaccatgtgaggcactaactgctttataaatattaacccatctaatctcatcacaatgaagatatgtactatgaggtaggtacttatctccattccattttacaggtaagaaaactaagttacagagattttaagtaaactgcccaagatcagacaactagtaggtagcaggagccaggccttgaactcaggcagtccagctccagagtttatttcttaatcactgcacaatgctacctcttttctcttttgtttcttttcctttttgctttcttcttcctctgcctaccctttgctgttggtgctctccagggctctgtctttagccttatcttactctattctctctccctgtgagatttcatccattcttagaggttttctgtagactctccagtgagatctctaagctggggagtaggaccattgtttctagccatatccctaatggccaccacagaacttggcacatacaagtgccaacaaagtatctgaatgaatgacaactcccagctctatctccagactggagctgagcttcgagacctggacactcaactgcccactagacatgcaaaggatgttagccaaataaggggaaagtggggataggataaagggggaaagagtattccaggaagaagagatcaagccctgccaactccatttccagagttgttaagctcatttctttcttccatctccactgacacagaccttccctcttacctggcccacagtcatggtctccctgactcatcttgctcccctccactcgacagtcctgtgccactcggtggcaaaatctaatccccttcctcccttgctgataaccctcagctggtttacaaggcctcctctcacattttgtgccgcagaacagagaaggaattgtaggcccctgaagaccccaagctgtctctcctcggaggcctttgctctaggggctggtccctgtgggctggcatgccccttctgcgagctcttaaagacactgctcaggtgtcaccgcctctggaatgcccaccctggggctcccagcttgaactgcctcctctccgcttcagtcctggtaaaggcgggccttaacaatcgttgagtcccagaaggcttcacttccgggctcgtctgcttcaccagccagtgagctcgggcgagcccgtccattctgggtctcactatccccacacctatccgtatgtagagtggcgctagctaggtcagacctcgcctgaagaccctgacatctgccgctgggcgacttggccccgcccccttgcctcccttgcgcggcaccgccccactcctccgtgctccccccgaggaaatggttcaaccgaagggcggtgccaaggtgcccgggccgctccgattggtcagggcgagccgtaccacggcggtggcgggggagcgcttcgtgggcagccggcgggctccgaggccgtgagcgcaaagcctcaggscccggctccctcctgagctgcgccgtgccaggccgcccgmcgggatgcartgggccgtgggycggcggtgggcgtgggccgcgctgctcctggctgtcgcagcggtgctgwcccaggtcgtctggctctggctgggtacgcagagcttcgtcttccagcgcgaagagatagcgcagttggcgcggcagtacgctggtgagcgggcagagggcagggaggggagmrgmcggcctgggagcggagcctagggttccgaaggcgccatccccggacctaggaccggtgccagccctgactccgccgcccctctgcctccgccagggctggaccacgagctggccttctctcgtctgatcgtggagctgcggcggctgcacccaggccacgtgctgcccgacgaggagctgcagtgggtgttcgtgaatgcgggtggctggatgggcgccatgtgccttctgcacgcctcgctgtccgagtatgtgctgctcttcggcaccgccttgggctcccgcggccactcgggtcagtgctagcggcgggccagamtgggagkctggggttgtaaaggggcccatgccctcctttctgatgtttggctgggccatgcaatggcgcgaataccctgatagtgtcatgtgcgattgtcactcagggcgctactgggctgagatctcggataccatcatctctggcaccttccaccagtggagagagggcaccaccaaaagtgaggtcttctacccaggtgggtaagaaggctcwgtcagagaagtgggtcccttcacgcggggggtgcccacaattgcggggagcatggcatggaaaaggagtgtgttgggggggccctcctcatggttcccttcctccaccctcccataagccacaggtgattggttggggcagagctgagggactcttgctttcctctgtcaaggggaaacaaaacataacaacttcctattaaacatgtctgatttttatctgcccaggcacaattttgcccagacagtattccaacaactgttggggagacaatgggtaatcagaaatgctctcaataccccaaaagacaagacagacagttatctttttatttgtttgttttttgagatggagtcttgctcagtcgcccaggctggcgtgcagtggcgcgatctcggctcactgcaagctccgcttcccgggttcacgccattctcctgcctcagcctcccgagtagctgggactacaggcgcccgccaccacgcccggctaattttttttgtatttttagtagagacggggtttcaccataccagccaggatggtctcgatctcctgacctcgtgatccacccgcctcggcctcccaaagttctgggattacaggtgtgagccaccgcgcccggccgctcccctgccttttttttttttttttttttttaatagtgaaacaatttacttaagaaatagagcactggacttcccagtatatttgaggttgggtgtatgcatcgcaggcatggtggttgaacagctacacttttcagataaagcctcacaagtcctggaactacccctttcctagctacaagcccagcccaggtttagctaaatttctgcttgattcgagtgaggcttgggagtgcagacttccatacagtgggtccatggccaagcgaggtagccgtccatggcttctccaaagagaggacgtgggtcagatgatgccacagaacatttaggagtggccatctgggccagcccagtgaggtagggctttttgtttttctgaagctccctctccattccccacccctagttagtccatgggccagggaagcagcagccatggaagggcagggcagagctggcttttcacttgggtgctccctgtcccccaggggagacggtagtacacgggcctggygaggcaacagctgtggagtgggggccaaacacatggatggtggagtacggccggggcgtcatcccatccaccctggccttygcgctggccgacactgtcttcagcacccaggacttcctcaccctcttctatactcttcgctcctatgctcggggcctccggcttgagctcaccacctacctctttggccaggacccttgaccagccaggcctgaaggaagacctgcggatagacaggagcgggcagrccckcacatatccacttgctggagcccatgtttacagacagggacatacaccatgcagatcctgagttcctgctgtatgagcagggatatccatgcttatgtatccaaacacagagacccatgggaacaaatgagacacatatagatactgagacctgtgtgtacagtaggaccatgcactcacacccatctggagagggagcccccggtataccaagggagccagttgtgttcagacacacacatcacagcttgactcactaactgaggcctttccatagctccacagcttcccacctcctccccaccaaaccggggttctagagttaaggatgggggagggtattatactgcctcagtctgactcctcaacccagcagcaatttgaggggatgagggggaagaggagctgccttttggaggcccccttcacctgcagctatgatgcccttccccttctcccctgtcctcaccatatgccttatccccattctactcccctgctatgcaagtgcccctgtggcttgtccccaaccccctcagcaacaaagctcagctggggaacgagagtaatttgaagaatgcttgaagtcagcgtcttccattccagaaagacccccattcttcctttgggggtatgatgtggaagctggtttcagcccaggacccaccactgaggagaggatctagacaggtgggcctaattccaaggggcccttcctggcctggagaaggccttttacacacacacaacacatacacacacacacacacacacacatatcacagttttcacacagcccctgctgcattctctgtccatctgtctgtttctattaataaagatttgttgatctgttccatctttttgtgtgtctgagcagccggaaaaagcctctgacccctggggacctcaggaagatctctctcttgctgtgtgattcatggtagattttttttttttttttttaagagatggggtctcctctgttgcccaggctggagtgcagtggcacagtgacatgatcatagctccctgcagccttgaactcatggtcccaaacaattttcctgccttagccttatgagtagctgggaccttatgagtagcaaatgccaccatgcctggttagctttttaaattttttttagaaatggggtcttgctatgttgccccggctgatctccagctcctggcctcaagtgatccttcctcagtctccagagtagctgagataacagaaacaagccaccatgcctgacaagacagtggcttttgactagtagggactatatacttgagtgtgccttagaatcacctaagacactctttgagtcagtaactcagactactgagctccattgccagagatcaacatgttcaataagatttcttccaacacagctgcgaatgccacaatttgagaaattcagggcagggtgacctggagtgtgtcttggtcttacctctgccggtgcaatcctgggaacagtcctaagaatcttcccctggctgaccacagggtggcagtgtgcacccactttttagctgaaaggcatagttctcagttggctttttttctgggctcccagagtagtgtctgcccccacccaaaacaggagatcctccagtctatggtctgccttctccaggacccaccactaatgtggaggctcccacaatgaaaggaaggcacactacctggcagtatgtgaggtaggtagggcctggactcaggtaaggcaagagaggtgcctaaagtgcattcattcccaaagaccctgctcttgccttaacttctgcattttacacaccaagcatgcctcaccctcgtcctggccctgttctgagggtaaggcatagatcactcctgcttctcaggattcctggtattttaaaaatggaagaaacatggttataaaaattagatattttaacattctaaagaacacaaatccgaaagactatatgtataactgcatttagaaacaacatcaaaagtcaatttgtgactgaatgtggagtccaggtaagttggccaacctgaaaccgactcttcacccccaataccaccgtgtcaggcatagtaggccctggctggctcctctccactcaccccaggttgcaaatccatgcctcctgggggattcctactctgtaacccagctctgcctctaggctcagagcccacatgtcagggatgacccagcttgccctgtcattcctcccaaacgtccacaaaggactaggctcccaggcccctcctaatctctgctgctaaatccgttggtcgtttcatagttcttagctttcatttgacacagggtaatcactctgaaggcactcttagtttctgggacactactcctccttaattgcctcctttactggaattttttttttttttttttttagtagagacaagagtttcaccacgttggccaggctggcctcaaactcctgacctcaagtaatccgcccatgttggccttacaaagtgctgggattacaggtgtgagccactgcacccagctactggattcctatctcctcattctctgactttcttgggcttagtcctcaaacctcttctgtttacaccccttcccaggtgattctgtgactttaccatctatatatcttgacttctaaatttttgtctcagcctaaatctctgccctgagcaccagagttggaaatcttactgccttcatctctactcaacatctccacttggctgtctaataggtatctcagacttgagatatccaaagcaaaccgaattctctcccagccccacaccaccaccatcctgtagtctttactgtgaattaagggcacctccatttacccagctgccc

SEQ ID NO: 14 - DNA, 361 bp, Homo sapiens
  misc_feature (53)..(54): n is a, c, g, or t
ttgcttcagagggtggaagccccaagccttgctgctgcacatcttagggagtnnggctcccagacaccctcacatggttctcagttgcctgagaatgccctgaaagctgaagggagcaaggtgctcttatttcttcagcatggaagattccacactcatgagttagaaggtttggagttagtccaattcaataagggaaaggacacacacacacacacacacacacacacaaagacataaacagacaacaaaacaggcaaaactaacantgatcacaaaanttatatgatttctgagcgctgtaagtntaagcagaaattaacaccaactggttgttattgctaactttagttgtttaaaa

SEQ ID NO: 15 - DNA, 395 bp, Homo sapiens
  misc_feature (93)..(93): n is a, c, g, or t
gatcaacaccattatttttaagtcaggtttttaaagccagagcctgccccatgctataggtttagcgccacatatcaaggaagaaggaaagantgtncttactttctgtaaaccaacttggcaaaggtaggtgggtgttgaaagagccacagtaaggctccttttgtggacacagagtgaattcaacaggaactgctctgaggcaatcacaggaaaaccctaatgtccagaaaggccacatggtgtccagcatagggaactacacacacacacacacacacacacacacacacacacggtgtccagcatagggaactacacacacacacacacacacacacacacacacacacacacactggctggaagtgtcagtgtgaggctccagctagatc

SEQ ID NO: 16 - DNA, 378 bp, Homo sapiens
  misc_feature (55)..(55): n is a, c, g, or t
gtttactcatttctaaaataagaataatacttaccacaacagttttaagtaaggnttaaatcaataagagattttaatatacatatatacaggatattatgtgatggaatccatttatctatctatctatctatctatctatctatcatctatctatctatcaagatagggagaaagagagagagagaagagatccttaagantaagctgtaggtaatattaggcatatttttggagtcagcagctaatctttcnctttaggantttccttgtggagtttatctaccncgggggcncaaaggntttnccngnctaantgcntntnantgntngggngnaatttccngctttgggncctttaggnaatccccnccnttt

SEQ ID NO: 17 - DNA, 446 bp, Homo sapiens
  misc_feature (261)..(261): n is a, c, g, or t
ttttttagtattactagatgtattagccagtgttctccagagaaacagaacttgtaggatatatagacatgatagatagatagatagatagatagatagatagatagatagatgatagatagacagatagatagaaagatagataatctattgtaaaaaaaagttaactcaagcagttattttggctgacatatcccaagacctgtagtcagcaagctggaaaggtaagagagccagtcttgtagttgtagtccgaagccnagtagacttgatacccagaggagtcaacattttggttgtccaaacatttcagggtccnaaggcaggaaaaatccaatgtcctagcttcaaaggcacttagacaggngggatttcntcttacttgngggagggtccagcctncttattttnttttagggnttttaaaggggtttgggaaagncccc

SEQ ID NO: 18 - DNA, 50 bp, Artificial Sequence
  Oligonucleotide for amplifying OPRS1 for recombinant expression
aaaagcttatggattacaaggatgacgacgataagcagtgggccgtgggc

SEQ ID NO: 19 - DNA, 27 bp, Artificial Sequence
  Oligonucleotide for amplifying Homo sapiens OPRS1 for recombinantexpression
aggatcctggtggggaggaggtgggaa

SEQ ID NO: 20 - DNA, 19 bp, Artificial Sequence
  Oligonucleotide for amplifying exons 2 and 3 of OPRS1
ggagcctagggttccgaag

SEQ ID NO: 21 - DNA, 23 bp, Artificial Sequence
  Oligonucleotide for amplifying exons 2 and 3 of OPRS1
caaccaatcacctgtggcttatg

SEQ ID NO: 22 - DNA, 20 bp, Artificial Sequence
  Oligonucleotide for amplifying OPRS1 mRNA
accatcatctctggcacctt

SEQ ID NO: 23 - DNA, 20 bp, Artificial Sequence
  Oligonucleotide for amplifying OPRS1 mRNA
ctccaccatccatgtgtttg

SEQ ID NO: 24 - DNA, 26 bp, Artificial Sequence
  Oligonucleotide for amplifying OPRS1 coding region
aaaagcttatgcagtgggccgtgggc

SEQ ID NO: 25 - DNA, 27 bp, Artificial Sequence
  Oligonucleotide for amplifying OPRS1 coding region
aggatcctggtggggaggaggtgggaa

SEQ ID NO: 26 - DNA, 50 bp, Artificial Sequence; identical to SEQ ID NO: 18
  Oligonucleotide for amplifying OPRS1 coding region with FLAG tag

SEQ ID NO: 27 - DNA, 27 bp, Artificial Sequence
  Oligonucleotide for amplifying OPRS1 coding region
aggatcctggtggggaggaggtgggaa